(19) United States
(12) Patent Application Publication
(10) Pub. No.: US 2016/0000126 A1
(43) Pub. Date: Jan. 7, 2016

(54) MODULATION OF SALTY TASTE PERCEPTION BY ALTERING THE FUNCTION OF BITTER- OR PKD2L1-EXPRESSING TASTE RECEPTOR CELLS

(71) Applicant: THE TRUSTEES OF COLUMBIA UNIVERSITY IN THE CITY OF NEW YORK, New york, NY (US)

(72) Inventors: Yuki OKA, New York, NY (US); Charles ZUKER, New York, NY (US)

(73) Assignee: The Trustees of Columbia University in the City of New York, New York, NY (US)

(21) Appl. No.: 14/767,566

(22) PCT Filed: Feb. 12, 2014
(86) PCT No.: PCT/US14/16028

§ 371 Date: Aug. 12, 2015

Related U.S. Application Data
(60) Provisional application No. 61/764,366, filed on Feb. 13, 2013.

(51) Int. Cl. A23L 1/22; G01N 33/50; C07D 285/135; A23L 1/237; C07C 331/22; C07D 495/04

(52) U.S. Cl. CPC A23L 1/22091; C07C 331/22; C07D 495/04; C07D 285/135; A23L 1/237; A23L 1/2375; G01N 33/5088; A23V 2002/00

(57) ABSTRACT

The current invention is in the field of taste and relates to methods and compositions to modulate the perceived taste of saltiness in food or food products where salty taste is desired. The methods and compositions relate to altering the activation and/or activity of the bitter-sensing taste receptor cells and the PKD2L1-expressing taste receptor cells. The invention also relates to food and food products containing agents and composition that alter the activation and/or activity of the bitter-sensing taste receptor cells and the PKD2L1-expressing taste receptor cells.

### CROSS REFERENCE TO RELATED APPLICATION

The present application claims priority to U.S. patent application Ser. No. 61/764,366 filed Feb. 13, 2013, which is hereby incorporated by reference in its entirety

### FIELD OF THE INVENTION

This invention is in the field of taste responses and is based on the discovery that taste responses to high concentrations of salt are mediated and modulated by the function of bitter- and PKD2L1 taste receptor cells.

### BACKGROUND OF THE INVENTION

Humans, as well as many mammals, categorize taste stimuli into five primary tastes: sweet, bitter, sour, salty, and umami (Chandrashekar et al. (2006); Yarmolinsky et al. (2009)). Sweet and umami are “good” tastes promoting consumption of nutritive food, while bitter and sour are “bad” tastes, alerting the organism to toxic food and preventing the consumption of food containing harmful substances (Yarmolinsky et al. (2009)). Salt can be “good’ or “bad”, depending on the concentration of the sodium and the need of the organism (Yarmolinsky et al. (2009)). However, natural taste stimuli are often complex, stimulating two or more taste modalities at once.

Taste stimuli are detected by taste receptor cells (“TRCs”) on the tongue and palate, each of which is narrowly tuned to detect chemical stimuli corresponding to one of the five primary taste modalities (Yarmolinsky et al. (2009)). These receptor cells are organized into taste buds, compact structures containing about 50-100 cells, and including all five of the taste receptor cell types, together with supporting cells and terminals of the afferent nerve fibers that convey information to the brainstem. Interestingly, components of diverse neurotransmitter and neuromodulatory systems, including transmitters, transporters and receptors, have been identified within taste buds (Dando and Roper (2012); Huang et al. (2012); Huang et al. (2005)). These findings support the possibility that lateral information flow within the taste bud might play an important role in gustatory processing (Herness and Zhao (2009); Roper (2007)).

Taste perception is initiated by the physical interaction of tastant molecules with specific receptor proteins located at the surface of the TRCs. Sweet and umami are sensed by heterodimeric G protein-coupled receptors (GPCRs) assembled by the combinatorial arrangement of T1R1, T1R2, and T1R3. Studies have shown that a heterodimeric receptor composed of T1R1 and T1R3 subunits (“T1R1+3”) is the mammalian umami receptor, and a heterodimeric receptor composed of T1R2 and T1R3 subunits (“T1R2+3”) is the mammalian sweet receptor, recognizing simple sugars, a wide range of artificial sweeteners, D-amino acids, and even some intensely sweet proteins (Yarmolinsky et al. (2009)).

Bitter tastes are sensed by a family of T2R bitter receptors, with a highly variable structure and few regions of extended conservation. This diversity in structure reflects the biological need of these receptors to recognize diverse chemicals (Yarmolinsky et al. (2009)).

Sour (acid)-sensing TRCs are characterized in their expression of PKD2L1. Carbonation, which also elicits a response in mammals, is recognized by the acid-sensing TRCs as well (Yarmolinsky et al. (2009)).

Taste signals from the taste buds are transmitted to neurons in the geniculate ganglion. Most taste responsive neurons in the geniculate ganglia were found to be narrowly tuned to a single primary taste.

Salty tastes are unique in that increasing salt concentration fundamentally transforms an innately appetitive stimulus into a powerfully aversive one (Beauchamp et al. (1990); Duncan (1962); Eylam and Spector (2005); Contreras (1989); Lindemann (2001)). This appetitive-aversive balance helps maintain appropriate salt consumption, and represents an important part of fluid and electrolyte homeostasis (Beauchamp et al. (1990); Duncan (1962); Contreras (1989); Chandrashekar et al. (2010)).

Cardiovascular diseases are the leading cause of death worldwide, and high blood pressure is a major risk factor (He and MacGregor (2009)). An estimated one in three Americans will develop high blood pressure (Vasan et al. (2002)), and a diet high in sodium is, at least in part, to blame. Sodium increases blood pressure because it holds excess fluid in the body, creating an added burden on the heart. A high sodium diet also may have other harmful health effects, including increased risk for stroke, heart failure, osteoporosis, stomach cancer and kidney disease (He and MacGregor (2009)). Moreover, the problem is starting early in America: 97 percent of children and adolescents eat too much salt, putting them at greater risk for cardiovascular diseases as they get older (Institute of Medicine: Dietary reference intakes for water, potassium, sodium chloride, and sulfate (2004)). The American Heart Association advises Americans to lower the amount of sodium they consume.

However, Americans have a strong appetite for a high salt diet, which needs to be satisfied without the potential negative effects of high sodium. One such solution to reduce the levels of sodium intake while preserving the salty taste that is craved in food. Another solution is the use of non-sodium salts, such as potassium chloride (KCl), as substitutes. Unfortunately non-sodium salts have a strong and unpalatable aftertaste that makes them undesirable substitutes.

Thus, there exists a need in the food industry of a way of reducing the sodium consumed by Americans, as well as those in other cultures, while still satisfying the desire for the taste of salt in food and food products.

### SUMMARY OF THE INVENTION

The present invention is based upon the surprising discovery that non-sodium salts such as potassium chloride (KCl), as well as high concentrations of sodium salts (NaCl at concentrations at about greater than 150 mM), innately activate two TRCs in the tongue, namely bitter and PKD2L1-expressing cells. It was also found that the membrane-bound carbonic anhydrase CA4, found on PKD2L1-expressing cells, is a target of high sodium and non-sodium salts. Thus, the perception of salty taste, in foods and food products, which contain either sodium or sodium substitutes, can be modulated by the activation or inhibition of bitter-sensing, and/or PKD2L1-expressing taste receptor cells and their concomitant pathways.

One embodiment of the current invention is a method to modulate the perception of salty taste by altering or modulating the activation or activity of bitter-sensing taste receptor cells (TRCs) and/or their concomitant pathway. This method can be accomplished by any agent or composition that alters or modulates the activation, expression and/or action of a molecule or the receptor of the molecule in the pathway.

A further embodiment of the present invention is an agent or composition which alters or modulates the activation or activity of bitter-sensing taste receptor cells and/or their concomitant pathway. Such agents or compositions can be added to food or food products in which high-salt taste is desired, including but not limited to, crackers, potato chips, corn chips, tortilla chips, sauces, and canned soups and vegetables. These agents or compositions will modulate the salty taste of the food or food product that contains sodium (NaCl), or a sodium substitute (e.g., KCl).

Yet another embodiment of the present invention is a composition comprising an agent or composition which alters or modulates the activation or activity of bitter-sensing taste receptor cells and/or their concomitant pathway in combination with a salt substitute. In a preferred embodiment, the salt substitute is potassium chloride.

Yet another embodiment of the present invention is a composition comprising an agent or composition which alters or modulates the activation or activity of bitter-sensing taste receptor cells and/or their concomitant pathway in combination with sodium chloride at various concentrations. The preferred concentration of NaCl is about greater than 150 mM.

Another embodiment of the current invention is a method to modulate the perception of salty taste by altering or modulating the activation or activity of PKD2L1-expressing, taste receptor cells (TRCs) and/or their concomitant pathway. This method can be accomplished by any agent or composition that would alter or modulate the activation, expression and/or action of a molecule or the receptor of the molecule in the pathway.

A further embodiment of the present invention is an agent or composition which alters or modulates the activation or activity of PKD2L1-expressing taste receptor cells and/or their concomitant pathway. Such agents or compositions can be added to food or food products in which high-salt taste is desired, including but not limited to, crackers, potato chips, corn chips, tortilla chips, sauces, and canned soups and vegetables. These agents or compositions will modulate the salty taste of the food or food product that contains either sodium (NaCl), or a sodium substitute (e.g., KCl).

Yet another embodiment of the present invention is a composition comprising an agent or composition which alters or modulates the activation or activity of PKD2L1-expressing taste receptor cells and/or their concomitant pathway in combination with a salt substitute. In a preferred embodiment, the salt substitute is potassium chloride.

Yet another embodiment of the present invention is a composition comprising an agent or composition which alters or modulates the activation or activity of PKD2L1-expressing taste receptor cells and/or their concomitant pathway in combination with sodium chloride at various concentrations. The preferred concentration of NaCl is about greater than 150 mM.

Since the carbonic anhydrase, CA4, plays a role in the activation of the PKD2L1-expressing TRCs, another embodiment of the current invention is a method to modulate the perception of salty taste by altering or modulating the activation or action of CA4.

Agents or compositions that alter or modulate the activation or action of CA4 are also an embodiment of the current invention. Such agents or compositions can be added to food or food products in which high-salt taste is desired, including but not limited to, crackers, potato chips, corn chips, tortilla chips, sauces, and canned soups and vegetables. These agents or compositions will modulate the salty taste of the food or food product that contains either sodium (NaCl), or a sodium substitute (e.g., KCl).

Yet another embodiment of the present invention is a composition comprising an agent or composition which alters or modulates the activation or activity of CA4 in combination with a salt substitute. In a preferred embodiment, the salt substitute is potassium chloride.

Yet another embodiment of the present invention is a composition comprising an agent or composition which alters or modulates the activation or activity of CA4 in combination with sodium chloride at various concentrations. The preferred concentration of NaCl is about greater than 150 mM.

Another embodiment of the current invention is a method to modulate the perception of salty taste by altering or modulating both the activation or activity of bitter-sensing taste receptor cells (TRCs) and/or their concomitant pathway, and the activation or activity of PKD2L1-expressing, taste receptor cells (TRCs) and/or their concomitant pathway. This method can be accomplished by any agent or composition or combination of agents or compositions that would alter or modulate the activation, expression and/or action of a molecule or the receptor of the molecule in the pathways.

A further embodiment of the present invention is an agent or agents or composition or compositions which modulates both the activation or activity of bitter-sensing taste receptor cells and/or their concomitant pathway, and the activation or activity of PKD2L1-expressing, taste receptor cells (TRCs) and/or their concomitant pathway. Such compositions may contain one or more agents that, alone or together, can alter or modulate both the activation of the bitter-sensing TRCs and the PKD2L1 TRCs. Such agents or compositions can be added to food or food products in which high-salt taste is desired, including but not limited to, crackers, potato chips, corn chips, tortilla chips, sauces, canned soups and vegetables. These agents or compositions will modulate the salty taste of the food or food product that contains either sodium (NaCl), or a sodium substitute (e.g., KCl).

Yet another embodiment of the present invention is a composition comprising an agent or agents or composition or compositions which alters or modulates both the activation or activity of bitter-sensing taste receptor cells and/or their concomitant pathway, and the activation or activity of PKD2L1-expressing, taste receptor cells (TRCs) and/or their concomitant pathway, in combination with a salt substitute. In a preferred embodiment, the salt substitute is potassium chloride.

Yet another embodiment of the present invention is a composition comprising an agent or agents or composition or compositions which alters or modulates both the activation or activity of bitter-sensing taste receptor cells and/or their concomitant pathway, and the activation or activity of PKD2L1-expressing, taste receptor cells (TRCs) and/or their concomitant pathway, in combination with sodium chloride at at various concentrations. The preferred concentration of NaCI is about greater than 150 mM.

A further embodiment of the present invention would be a method and/or assay for screening to identify agents for modulating the perception of salty taste in a food or food product by altering or modulating the activation or activity of bitter-sensing taste receptor cells (TRCs) and/or their concomitant pathway. Such a method of, or assay for, screening would comprise:
a. stimulating the tongue of an animal with a high concentration of sodium chloride or a sodium substitute, wherein neural responses to taste of the animal can be recorded;
b. recording the neural response of the animal to the high concentration of sodium chloride or sodium substitute;
c. stimulating the tongue of an animal with a bitter taste stimulant, wherein neural responses to taste of the animal can be recorded;
d. recording the neural response of the animal to the bitter taste stimulant;
e. administering the agent to the animal;
f. repeating steps a.-d.; and
g. comparing the second neural responses of the animal to the first neural responses of the animal;
wherein if the second neural responses of the animal to the sodium chloride or the sodium substitute, and the bitter taste stimulant, are less than the first neural responses of the animal to the sodium chloride or the sodium substitute, and the bitter taste stimulant, the agent is altering or modulating the activation or activity of bitter-sensing taste receptor cells (TRCs) and/or their concomitant pathway by the high concentration of sodium or the sodium substitute, and can be used to modulate the perception of salty taste in a food or food product.
   The preferred sodium substitute to be used in the method or assay is potassium chloride.

A further embodiment of the present invention would be a method and/or an assay for screening to identify agents for modulating the perception of salty taste in a food or food product by altering or modulating the activation or activity of PKD2L1-expressing taste receptor cells (TRCs) and/or their concomitant pathway. Such a method of, or assay for, screening would comprise:
a. stimulating the tongue of an animal with a high concentration of sodium chloride or sodium substitute, wherein neural responses to taste of the animal can be recorded;
b. recording the neural response of the animal to the high concentration of sodium chloride or sodium substitute;
c. stimulating the tongue of the animal with an acid taste stimulant, wherein neural responses to taste of the animal can be recorded;
d. recording the neural response of the animal to the acid taste stimulant;
e. administering the agent to the animal;
f. repeating steps a.-d.; and
g. comparing the second neural responses of the animal to the first neural responses;
wherein if the second neural responses of the animal to the sodium chloride or the sodium substitute, and the acid taste stimulant, are less than the first neural responses of the animal to the sodium chloride or the sodium substitute and the acid taste stimulant, the agent is altering or modulating the activation or activity of PKD2L1-expressing taste receptor cells (TRCs) and/or their concomitant pathway by the high concentration of sodium or the sodium substitute, and can be used to modulate the perception of salty taste in a food or food product.
   The preferred sodium substitute to be used in the method or assay is potassium chloride.

A further embodiment of the present invention would be a method or an assay for screening to identify agents that modulate the attraction or aversion to high concentration of sodium or sodium substitutes. Such a method of, or assay for screening, would comprise:
a. treating a cohort of animals to conditions favoring salt-aversion or salt attraction;
b. administering the agent to some of the cohort of animals;
c. recording the behavior of the entire cohort of animals in response to sodium or sodium substitutes; and
d. comparing the aversion or attraction to the sodium or sodium substitutes of the animals administered the agent, to those animals who were not administered the agent;
wherein if the animals which received the agent have a different behavioral response to the sodium or sodium substitute, i.e., a greater aversion or attraction, the agent is modulating the attraction or aversion to sodium or sodium substitutes.

If the agent is known to modulate the activation or activity of bitter-sensing taste receptor cells (TRCs) and/or their concomitant pathway, then the agent is considered to be modulating the aversion to high sodium or sodium substitutes via the bitter-sensing TRCs.

If the agent is known to modulate the activation or activity of PKD2L1-expressing taste receptor cells (TRCs) and/or their concomitant pathway, then the agent is considered to be modulating the aversion to high sodium or sodium substitutes via the acid-sensing TRCs.

A further embodiment of the present invention would be a method and/or an assay for screening to identify agents for modulating the perception of salty taste in a food or food product that alter or modulate the activation and/or action of the enzyme CA4, or those which modulate the activity of PKD2L1-expressing TRCs. Such a method of, or assay for, screening would comprise:
a. stimulating the tongue of an animal with a high concentration of sodium chloride or sodium substitute, wherein neural responses to taste of the animal can be recorded;
b. recording the neural response of the animal to the high concentration of sodium chloride or sodium substitute;
c. stimulating the tongue of the animal with an agent or composition with a pH of less than 7.0, wherein neural responses to taste of the animal can be recorded;
d. recording the neural response of the animal to the agent or composition with a pH of less than 7.0;
e. administering the test agent to the animal;
f. repeating steps a.-d.; and
g. comparing the second neural responses of the animal to the first neural responses;
wherein if the second neural responses of the animal to the sodium chloride or the sodium substitute, and the agent or composition with a pH of less than 7.0, are less than the first neural responses of the animal to the sodium chloride or the sodium substitute and the agent or composition with a pH of less than 7.0, the test agent is altering the activation and/or action of the enzyme CA4, by the high concentration of sodium or the sodium substitute, and can be used to modulate the perception of salty taste in a food or food product.
   The preferred sodium substitute to be used in the method or assay is potassium chloride.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 depicts images of the results of double label in situ hybridization of the expression in the sapphire transgene in the taste buds of T2R32-Sapphire mice. FIG. 1A (left panel) show the expression of sapphire, FIG. 1A (middle panel) shows the expression of a mix of 20 T2Rs, and FIG. 1A (right panel) shows the co-expression of both. FIG. 1B shows the expression of the sapphire gene and PKD1L3, and FIG. 1C shows the expression of the sapphire gene and T1R3.

FIG. 2 shows the integrated chorda tympani responses of wild type mice to taste stimuli before (indicated by the − above the trace) and after (indicated by the + above the trace) application of AITC. Representative responses from multiple animals are shown.

FIG. 3 shows a graph of the quantification of integrated chorda tympani recordings before (white bars) and after (grey bars) AITC treatment. Tastants used were: 20 mM citric acid, Sour; 20 mM AcesulfameK, Sweet; 50 mM MPG +0.5 mM IMP, Umami; 60 mM NaCl, Low-salt; 0.1 mM cycloheximide, Bitter; and 120, 250 and 500 mM KCl or NaCl+10 mM amiloride, High-salt. Data were normalized to the response of 20 mM citric acid and are means±s.e.m, n≧3 mice. Student's t-test, P<0.05.

FIG. 4 depicts representative chorda tympani recordings showing responses to: (A) 5 minutes exposure of the tongue to 3 mM AITC; (B) without AITC treatment, and 1 and 30 minute AITC treatment and exposure to bitter tastant cycloheximide (1 mM); (C) with AITC treatment (indicated by the + above the trace) and without AITC treatment (indicated by the − above the trace), and exposure to bitter tastants, 0.1 mM cycloheximide, 10 mM denatonium, and 10 mM quinine, and sour taste, 20 mM citric acid; and (D) bitter and high-salt exposure in the presence (indicated by the + above the trace) and absence (indicated by the − above the trace) of AITC in TRPA1-KO mice.

FIG. 5 depicts calcium imaging of taste cell responses. A taste bud overlaid with Sapphire fluorescence (dotted circle, left) and pseudo-colored images depicting taste responses to high-salt, bitter and sour stimuli (right panels); scale bar, 10 μm. Below the imaging panels are representative ΔF/F traces for these tastants from three additional Sapphire-positive cells.

FIG. 6A is a plot of response amplitudes (ΔF/F) for 500 mM KCl (black bars) and bitter (a mix of 10 mM denatonium, 1 mM quinine and 1 mM cycloheximide, grey bars) in responding T2R32-sapphire cells. FIG. 6B is a graph of dF/F responses of T2R32-sapphire cells shown in FIG. 6A to KCl, bitter and sour stimuli (mean±s.e.m, n=16 for bitter and KCl, and 14 for sour).

FIG. 7A shows representative chorda tympani responses from control (WT), TRPM5-KO, PLCβ2-KO (PLC-KO) and T2R32-PLCβ2 (T2R-PLC) rescue mice before (−) and after (+) application of AITC. FIG. 7B is a graph of a quantification of normalized responses, before (white bars) and after (grey bars) application of AITC (mean±s.e.m, n≧3 animals). (Student's t-test, P<0.05).

FIG. 8A shows integrated chorda tympani recordings showing the responses of wild-type, PKD2L1-TeNT and TRPM5-KO/PKD2L1-TeNT double mutant animals to various tastes stimulants, including high salt. FIGS. 8B-D are graphs quantifying the responses shown in FIG. 8A (Student's t-test, P<0.001). Data in FIGS. 8B-D were normalized to the response of 60 mM NaCl and are means±s.e.m, n≧3 animals.

FIG. 9 show integrated chorda tympani recordings of PKD2L1-TeNT mice to various taste stimuli in the presence of AITC (lower panel) or the absence of AITC (upper panel). Tastants used were citric acid (20 mM), acesulfame K (AceK, 20 mM), monopotassium glutamate+IMP (MPG, 50 mM+IMP 0.5 mM), cycloheximide (Cyx, 0.1 mM) and NaCl and KCl at indicated concentrations (mM).

FIG. 10 shows graphs of the results of immediate lick assays used to measure behavioral responses to KCl (aversion, FIG. 10A) and NaCI (attractive, FIG. 10B after sodium depletion) in control mice (WT, solid black line), TRPM5-KO/PKD2L1-TeNT double mutant animals (grey line), and single mutants (dotted lines). Two-way ANOVA with post hoc test, P<0.001. Values are means±s.e.m., n>6 mice.

FIG. 11 shows a graph of the results of immediate lick assays used to measure behavioral responses to NaCl in the presence of 30 mM amiloride in control mice (WT, solid black line), TRPM5-KO/PKD2L1-TeNT double mutant animals (grey line), and single mutants (dotted lines). Two-way ANOVA with post hoc tests at individual salt concentrations revealed significant differences between double mutants and other genotypes at 250 mM NaCl (P<0.05) and at 500 mM NaCl (P<0.01). Values are means±s.e.m. n>6 mice for each point; data represent the percentage of licks relative to water licks.

FIG. 12A shows the quantification of integrated chorda tympani nerve responses in TRPM5-KO/PKD2L1-TeNT double mutant mice to varying concentrations of KCl. Data were normalized to the response of 60 mM NaCl and are means±s.e.m, n=3 animals. FIG. 12B depicts the results of immediate lick assays used to measure salt attraction to low concentrations in control and double mutant mice to various concentrations of KCl (WT, black circles; double mutant animals, grey circles) and NaCl (WT, black triangle; double mutant animals, grey triangle). Values are means±s.e.m., n=5 mice.

FIG. 13A shows graphs quantifying chorda tympani responses from control (WT), heterozygous (CA4 +/−, white bars) and homozygous CA4 −/− mice (grey bar) to KCl after AITC treatment at various concentrations. Student's t-test, P<0.05; values are means±s.e.m., n>3 mice. FIG. 13B shows quantification of chorda tympani responses to 500 mM KCl or 100 mM CaCl₂ before (white bars) and after addition of 30 mM KHCO₃ (grey bars) of TRPM5-KO mice. P<0.05, Student's t-test; values are means±s.e.m., n≧3 mice). FIG. 13C depicts representative chorda tympani responses. Upper panel shows responses from TRPM5-KO mice, and the lower panel responses from PKD2L1-TeNT mice, to various salts, before (left black traces of pair), and after (right grey traces of pair) treatment of the tongue with DZA.

FIG. 14A shows the quantitation of normalized chorda tympani responses to 500 mM KCl and 100 mM CaCl₂ at a pH of 7.4 (normal artificial saliva) and a pH of 5.5 in TRPM5-KO mice (* indicates significance P<0.05, Student's t-test). FIG. 14B shows the quantitation of normalized chorda tympani responses to 500 mM KCl and 100 mM CaCl₂ at a pH of 7.4 (normal artificial saliva) and a pH of 5.5 in PKD2L1-TeNT mice. Data were normalized to the response of 60 mM NaCl and are means±s.e.m, n=3 animals.

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “modulate” and the like, and the term “alter” and the like, as used herein means to change, either positively or negatively, either to enhance or increase or activate, or to suppress or eliminate or decrease or inhibit.

The term “agent” as used herein means a substance that produces or is capable of producing an effect and would include, but is not limited to, chemicals, pharmaceuticals, biologics, small organic molecules, antibodies, nucleic acids, peptides, and proteins.

The terms “screen” and “screening” and the like as used herein means to test an agent to determine if it has a particular action or efficacy.

The terms “identification”, “identify”, “identifying” and the like as used herein means to test agents and their ability to have a particular action or efficacy.

High-Salt Shares Common Pathways with Bitter and Sour Tastes,

As discussed above, gustatory responses to primary taste qualities are mediated by distinct, segregated populations of taste receptor cells. However, natural taste stimuli are often complex, stimulating two or more taste modalities at once.

Sodium is an essential ion, and as such animals have evolved dedicated salt-sensing systems, including prominent detectors in the taste system. Salt taste in mammals can trigger two opposing behavioral responses. On the one hand, low concentrations of salt (less than 100 mM NaCl, referred to as “low-salt”) are generally appetitive and elicit behavioral attraction. On the other hand, high concentrations (greater than 300 mM, referred as “high-salt”) are aversive, and provoke strong behavioral rejection. Notably, the attractive salt pathway is selectively responsive to sodium (underscoring the key requirement of NaCl in the diet), while the aversive one functions as a non-selective detector for a wide range of salts (Beauchamp et al. (1990); Duncan (1962); Contreras (1989); Lindemann (2001)).

It has been previously shown that the appetitive responses to NaCl are mediated by taste receptor cells expressing the epithelial sodium channel, ENaC, while the cellular substrate for salt aversion was unknown (Chandrashekar et al. (2010)). Also, for years, the sensitivity of ENaC to the diuretic amiloride has been used as a powerful means to block ENaC function and separate the contributions of the appetitive and aversive salt pathways (Chandrashekar et al. (2010); Doolin and Gilbertson (1996); Halpern (1998); Heck et al. (1984); Hettinger and Frank (1990); Spector et al. (1996)).

Based upon these findings, it was hypothesized that identifying an equivalent pharmacological blocker for the high-salt sensing pathway would provide a valuable tool for dissecting the cellular basis of the high-salt sensing pathway as well as a tool for modifying the aversion to non-sodium salts and the attraction to sodium.

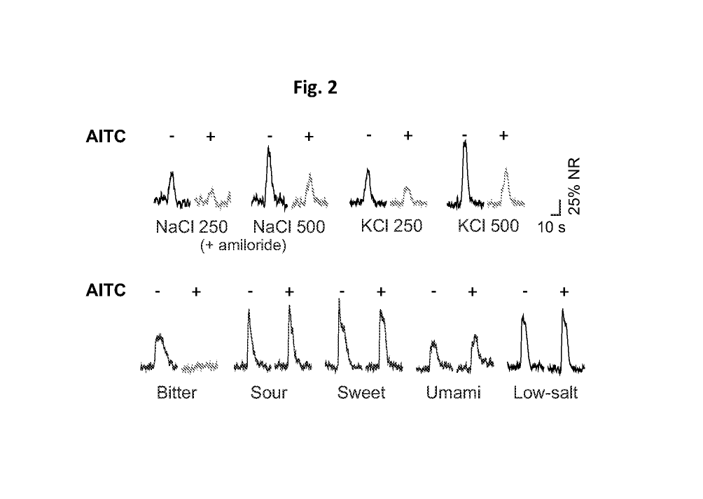

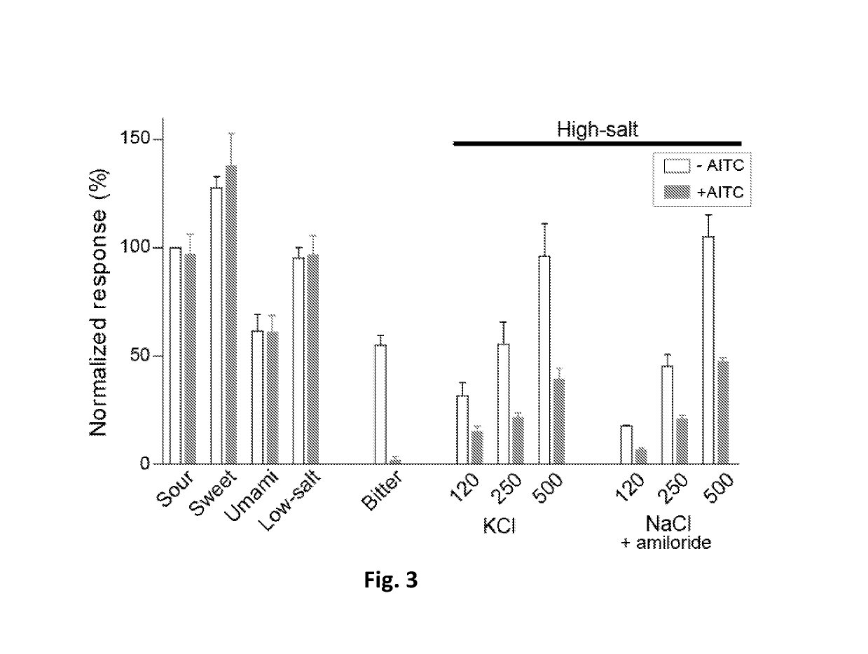

The chorda tympani, i.e., neural, taste responses in the presence and absence of various compounds that are known to affect ion channel function were performed, and it was found that allyl isothiocyanate (AITC), a component of mustard oil (and its source of pungency) significantly suppressed high sodium responses, without affecting responses to low concentration of NaCl. AITC also suppressed responses to the non-sodium salt, KCl, which selectively activates the high-salt pathway (Example 3; FIGS. 2 and 3). Interestingly, AITC also inhibited the responses to bitter stimuli without significantly impacting other taste modalities (Example 3; FIGS. 2-4). These results suggested that the bitter taste receptor cells may be a target of AITC, and a constituent of the high-salt sensing pathway.

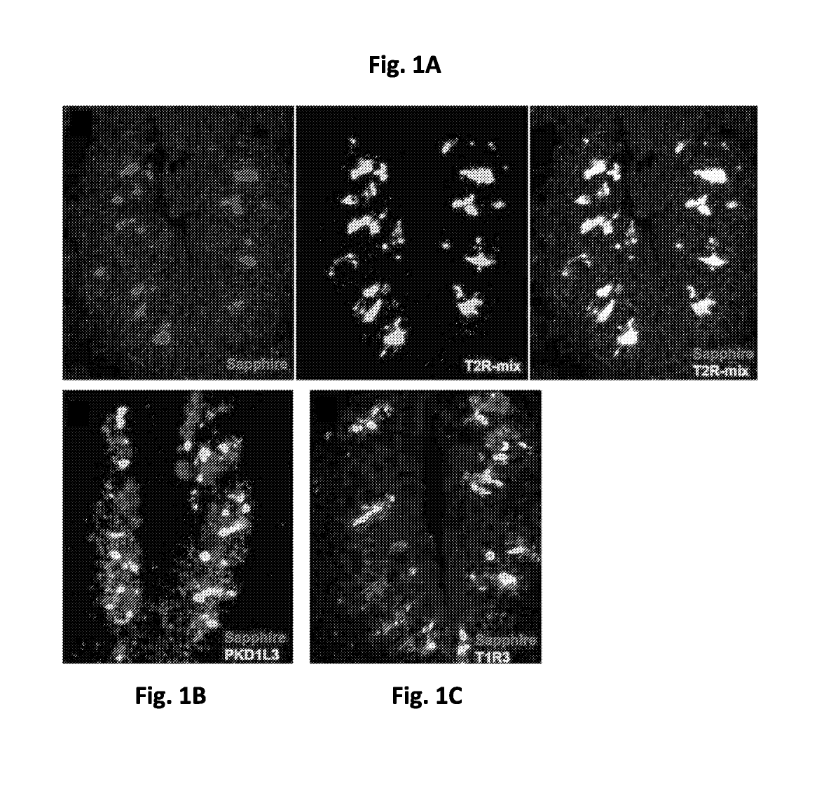

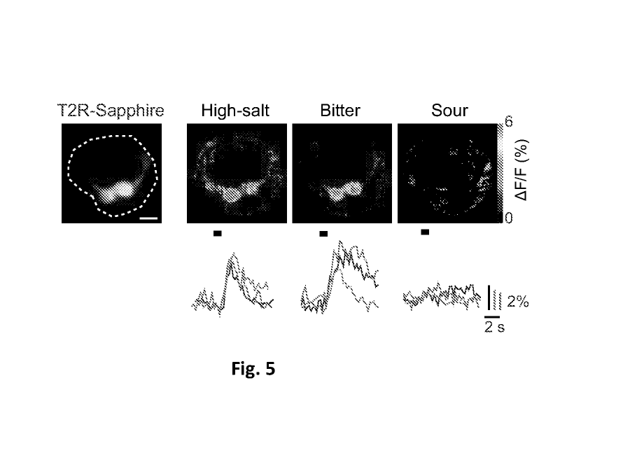

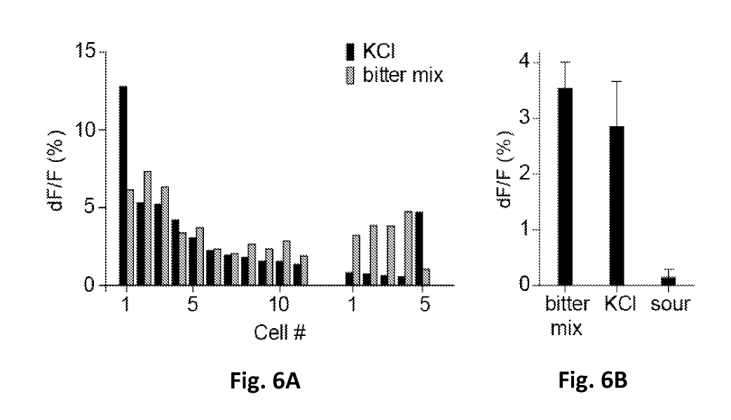

The next step was to determine if bitter-sensing cells are activated by high-salt stimuli. By using mice that selectively express bitter taste receptors (Example 2; FIG. 1) and a method that allows the functional imaging of taste receptor cells in response to tastant stimulation with single cell resolution, it was found that high concentration of salt activates bitter TRCs (Example 4; FIGS. 5 and 6). These animals responded to bitter stimuli, and high-salt but not sour stimuli.

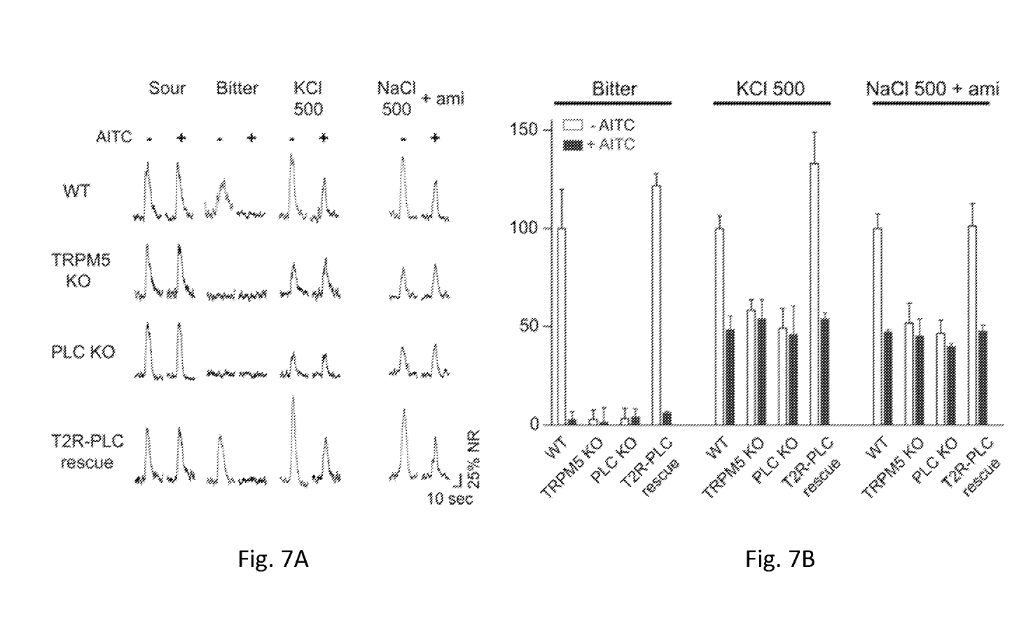

The finding that high-salt activates bitter-sensing cells, and the observation that high-salt and bitter stimuli are both blocked by AITC, suggest that bitter and high-salt may share a common pathway (e.g. through the T2R pathway). If so, it would be expected that TRPM5 or PLC32 knockout mice, which lack key components for bitter taste signaling, would also defective in high-salt sensing. Indeed, it was shown that this was the case: the nerve responses of the knockout animals to high-salt are significantly reduced, and they are no longer sensitive to AITC. Additionally, when PLC function was restored only to bitter taste receptor cells of PLCβ2 knockout mice, the electrophysiological responses to both bitter and high-salt were restored to levels indistinguishable from those in wild type mice. These results demonstrate that bitter sensing cells mediate the PLCβ2-dependent high-salt responses, and support the proposal that the aversion to high-salt is mediated, at least in part, by activation of the bitter-sensing pathway (Example 5; FIG. 7).

These findings show that AITC and TRPM5/PLCβ2 knockouts eliminate only about 50% of the high-salt neural responses (Example 5; FIG. 7). Not surprisingly, these animals still retain strong behavioral aversion to high-salts (Zhang et al. (2003)). Thus, it was hypothesized that other cells are mediating the remaining neural responses and behavior. Given that high-salt is strongly aversive, and recruits one of the primary aversive taste pathways, it was hypothesized that sour, the other principal aversive pathway would mediate the remaining responses.

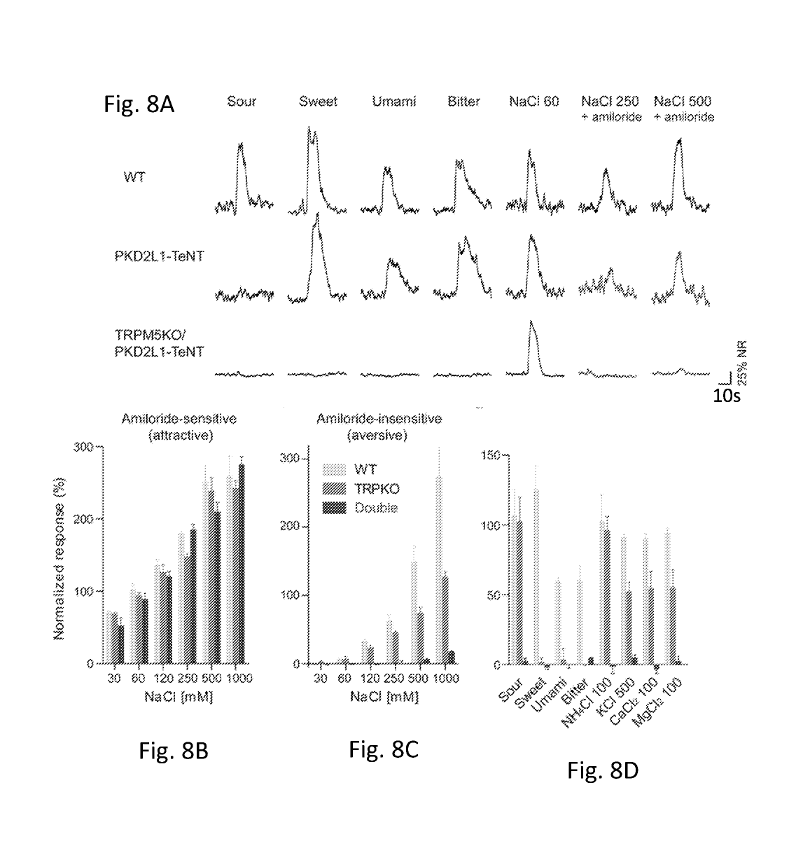

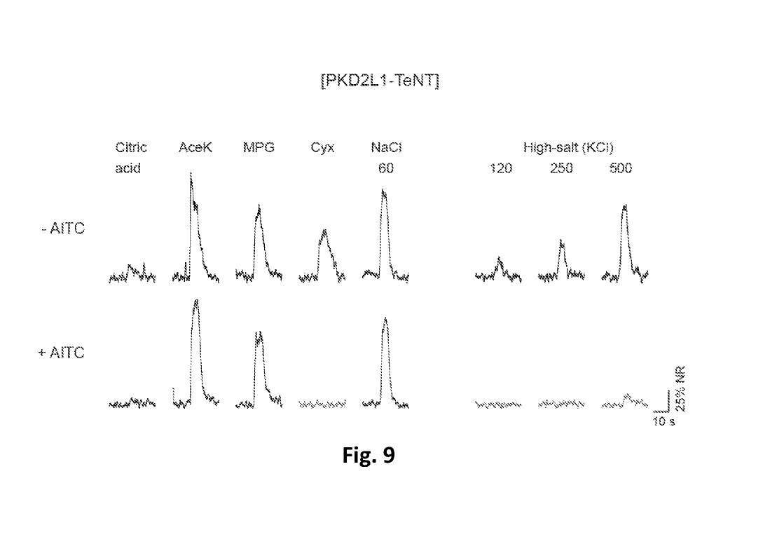

To examine the involvement of sour-sensing cells in high-salt detection, mice engineered to have inactivated sour TRCs (i.e. PKD2L1-expressing cells) were assayed for their tastant-evoked neural activity in response to salt stimulation. As shown previously (Chandrashekar et al. (2009)), silencing PKD2L1-expressing cells eliminates acid evoked taste responses (Example 6; FIG. 8A). However, surprisingly, these animals also display a major reduction in their high-salt electrophysiological responses, and further treatment with AITC effectively abolished their remaining high-salt (KCl) responses (Example 6; FIG. 9).

Because it was considered that high-salt taste responses are most likely mediated by the combined action of bitter and sour-sensing cells, and genetically blocking both pathways should abolish high-salt responses. Indeed, double mutant mice lacking components to both the sour and bitter taste pathways, exhibit a near complete loss of electrophysiological taste responses to a variety of high-salts, including concentrations of NaCl as high as 1000 mM (Example 6; FIG. 8).

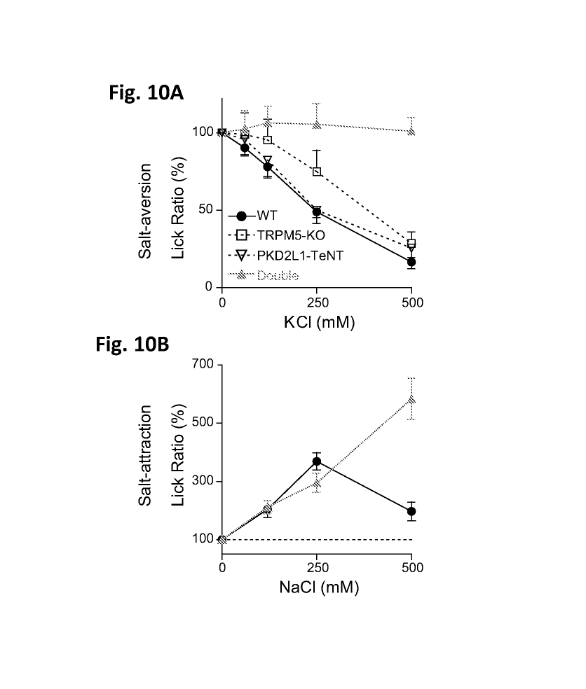

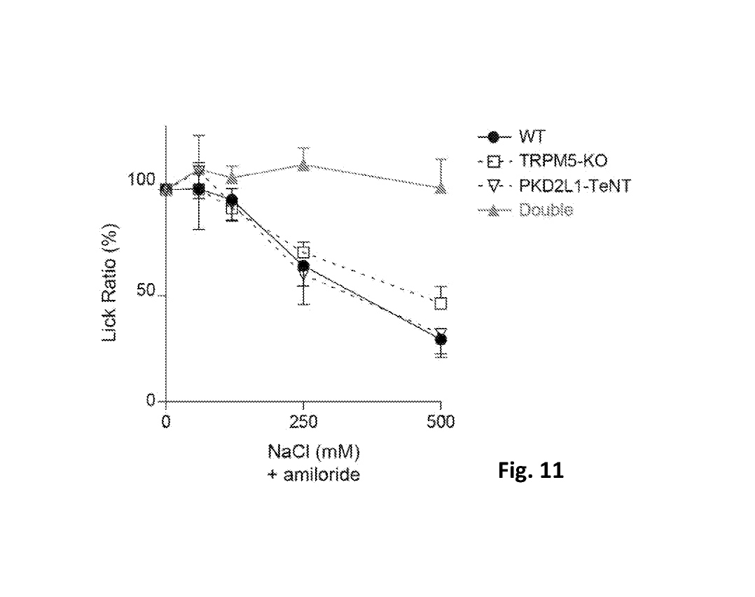

It was further found that mice with single mutations, either in the bitter- or sour-tasting pathway, retain a strong behavioral aversion to high salt, demonstrating that activation of either pathway on its own is sufficient to trigger behavioral rejection to salt (Example 7; FIGS. 10 and 11).

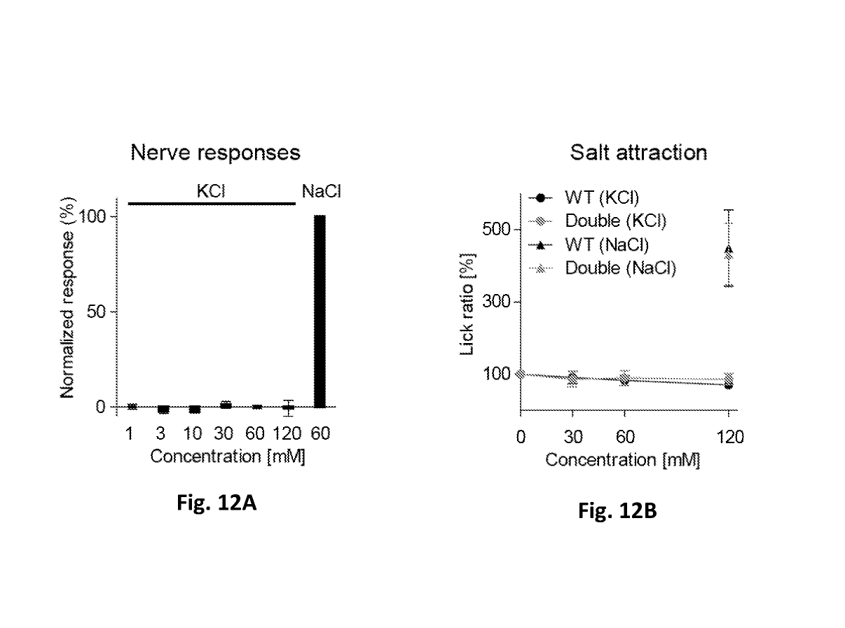

Surprisingly, double mutant animals, with mutations in both pathways, exhibit no behavioral salt aversion even at concentrations where controls are strongly repelled. Remarkably, these double mutants are not simply indifferent to high-salt, but now exhibit unimpeded attraction, even to exceedingly high concentrations of salt (e.g. levels equivalent to ocean water) (Example 7; FIGS. 10 and 12).

Thus, under normal conditions the appetitive-aversive balance to salt, which collectively tunes the animal's behavioral response to sodium salts, must be orchestrated by the combined activity of the attractive ENaC pathway (which remains in the bitter/sour double mutants) and the repulsive T2R and sour pathways.

It is not known how high-salt activates the bitter and sour taste receptor cells. Without being bound by any theories, it is reasoned that because the primary effectors of T2R signaling in bitter cells, PLCβ2 and TRPM5, are also required for high-salt sensing by the bitter cells, either a signaling component in bitter cells (for example an ion channel), or one or more of the three dozen T2R-receptors, might be sensitive to high concentrations of salt and perhaps cause the transition between the receptor's inactive and active state. (Gao et al. (2000); Liu et al (2012)).

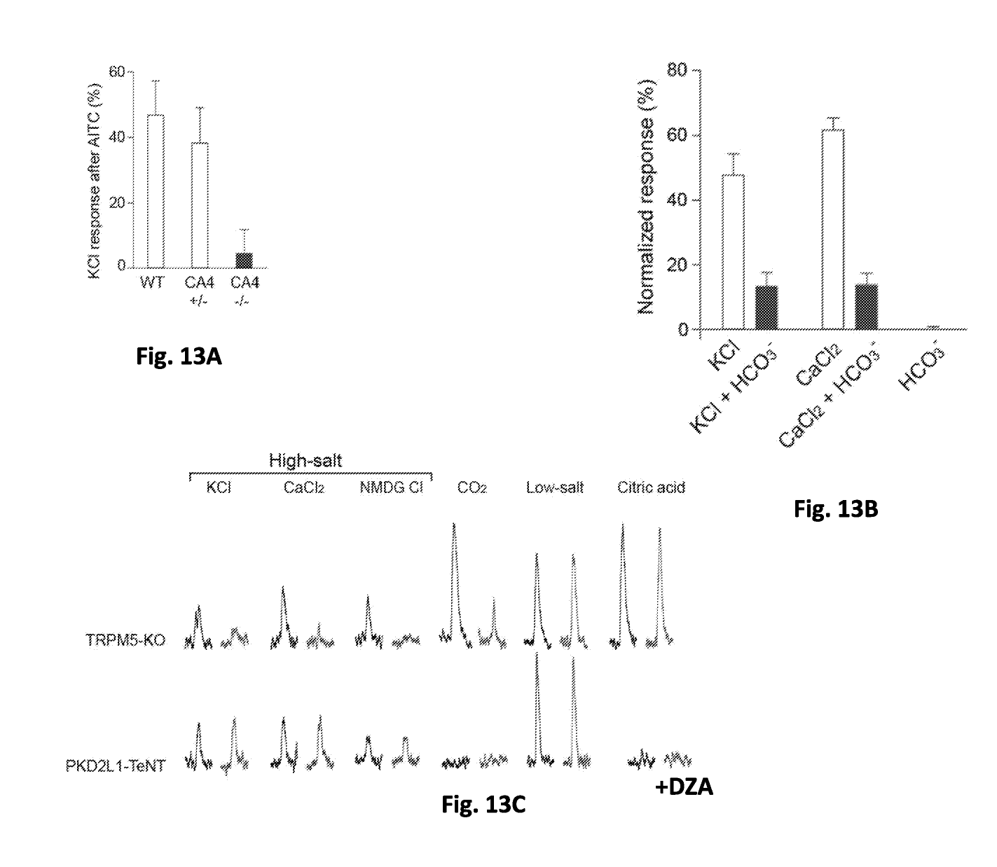

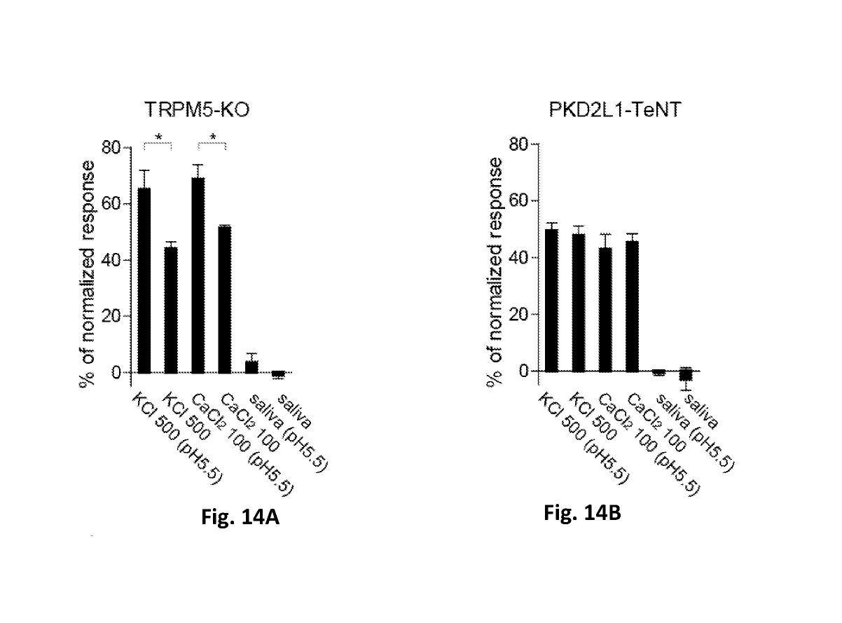

A salient feature of sour cells is the prominent expression of carbonic anhydrase 4 (CA4), a membrane-bound isoform of carbonic anhydrase (Yamamoto et al. (2003)). CA4 is likely involved in buffering the pH around taste receptor cells (CO2+H₂O⇄HCO3−+H+), and therefore its activity may directly impact local proton concentration and acid sensing. Notably, carbonic anhydrases are known to be sensitive to high ionic strength environments, with high-salt concentrations strongly inhibiting their enzymatic activity (Zhu et al. (1990); Baird et al. (1997)). This raises the possibility that CA4 may function as a “translator” of external salts into local pH changes, and thus operate as an important component of high-salt receptor in sour-sensing cells. Supporting this proposal, the results herein demonstrate that pharmacological inhibition of tongue carbonic anhydrases, or the knockout of CA4, greatly impairs high-salt sensing by the sour taste receptor cells (Examples 8 and 9; FIGS. 13 and 14).

Taken together, the results herein demonstrate that salts activate three different classes of TRCs: the appetitive responses are mediated through the sodium selective ENaC pathway (Chandrashekar et al. (2006)), while the rejection of high-salt results from the recruitment of the sour and bitter pathways. At a cellular level, these results explain the conundrum of a “valence change” by reducing the problem to simply having distinct cell types with well-defined but opposing valences responding to salt. At a physiological level, these results provide a simple explanation for the longstanding observation that bitter and sour afferent fibers behave as “generalists”, responding not only to bitter and acid stimuli, but also to a variety of salts (Hellekant et al (1997)). The finding that T2R- and PKD2L1-cells are also activated by high-salt does not imply a change in the logic of taste coding, or in the valence/quality encoded by these TRCs. In fact, these results show that the bitterness (Breslin and Beauchamp (1995)), and “ionic” taste associated with high concentrations of non-sodium salts in humans may indeed be mediated by the concurrent activation of T2R- and PKD2L1-expressing cells.

High salt does not taste like a mix of sour and bitter because sourness represents the detection of protons by at least two separate signaling pathways in the oral cavity: taste (via PKD2L1-cells) and non-taste (via TRPVI-, ASIC-, etc.) (Hallock et al. (2009); Ohkuri et al. (2012); Ugawa et al. (2005)), thus it is hypothesized that the activation of PKD2L1 cells, in the absence of the non-taste acid sensing pathway may instead evoke the ionic taste characteristic of high concentrations of non-sodium salts. This proposal recasts PKD2L1 cells, and their corresponding (labeled) neural line as sensors of ions (protons, potassium, etc), orchestrating different percepts whether activated alone (e.g. ionic taste) or in combination: PKD2L1+nontaste acid sensors=sourness, while PKD2L1+T2Rs=the taste of KCl and other non-sodium salts.

These findings now provide a novel strategy for modulating the perception of salty taste in food and food products with NaCl or sodium substitutes such as KCl by targeting the bitter- and sour-sensing taste receptor cells.

### Methods of Use, Agents, Compositions and Products

Based upon the findings summarized above, and set forth in detail in the Examples, a method of modulating perceived salty taste by altering or modulating the activity and/or activation of the bitter-sensing and/or the PKD2L1-expressing taste receptor cells in the presence of a salt stimulus is contemplated by the current invention. This method can be accomplished in several ways. Agents and compositions that can be used in these methods are also contemplated by the current invention.

One method for modulating perceived salty taste is to alter the activation and/or activity of bitter-sensing taste receptor cells and their concomitant pathway. This method can be accomplished by any agent that would modulate the activation, expression and/or action of a molecule or the receptor of the molecule in the pathway. Such agents include but are not limited to chemicals, phytochemicals, pharmaceuticals, biologics, small organic molecules, antibodies, nucleic acids, peptides, and proteins. Agents that modulate this activation include but are not limited to, allyl isothiocyanate (AITC). It is contemplated that these agents would be ingested by, or administered a subject which is ingesting the food or food product and would be ingested or administered before, during, or slightly after the ingestion of a food or food product in which the salty taste is to be modulated. AITC can be administered in an amount ranging from about 1 to 10 mM with 3 mM being most preferred.

Another embodiment of the current invention is a composition comprising an agent that modulates the activation and/or activity of bitter-sensing taste receptor cells and their concomitant pathway, alone or in combination with NaCl. The concentration of NaCl can vary.

Another embodiment is a composition comprising an agent that modulates the activation and/or activity of bitter-sensing taste receptor cells and their concomitant pathway, alone or in combination with a sodium substitute, including but not limited to potassium chloride, magnesium chloride, and calcium chloride.

Another embodiment of the present invention is an agent or composition that modulates the activation and/or activity of bitter-sensing taste receptor cells and their concomitant pathway added to a food or food products in which high-salt taste is desired, including but not limited to, crackers, potato chips, corn chips, tortilla chips, sauces, and canned soups and vegetables. These compositions will modulate the salty taste of the food or food product that contains either sodium (NaCl), or a sodium substitute (e.g., KCl).

A food or food product containing an agent or composition that modulates the activation and/or activity of bitter-sensing taste receptor cells and their concomitant pathway are also part of the current invention. One such embodiment is a food or food product in which high salt is desired comprising either NaCl or a sodium substitute, and AITC. In a preferred embodiment the sodium substitute is potassium chloride. In another preferred embodiment, the AITC is in the food or food product in an amount ranging from about 1 to 10 mM.

It will be understood by those of skill in the art that one aspect of the invention allows the use of less sodium and/or a sodium substitute in a food or food product where a salty taste is desirable. This can be accomplished by modulating the function of the bitter-sensing TRCs such that the subject perceives high salt where there is a lower amount of sodium and/or a sodium substitute. Thus, because less sodium or a substitute can be used in the food or food product, and the same desirable salty taste obtained, the food or food product is healthier but still satisfies the subject's desire for high salt taste.

It will also be understood by those of skill in the art that the invention allows the use of less sodium by using sodium substitutes and modulating the activity of bitter-sensing TRCs in a food or food product where a salty taste is desirable. This can be accomplished by simultaneously modulating the function of the bitter-sensing TRCs that normally respond to high salt such that the subject perceives high salt with the use of a substitute, but without the aversive responses associated with high concentrations of sodium and salt substitutes. Thus, because less sodium, i.e., a sodium substitute, can be used in the food or food product, without the substitute evoking a bad aftertaste, and the same desirable salty taste obtained, the food or food product is healthier but still satisfies the subject's desire for high salt taste.

Because high-salt also activates the PKD2L1-expressing taste receptor cells, the second method for modulating perceived salty taste is to alter the activation and/or activity of PKD2L1-expressing taste receptor cells and their concomitant pathway. This method can be accomplished by any agent that would modulate the activation, expression and/or action of a molecule or the receptor of the molecule in the pathway. Such agents include but are not limited to chemicals, phytochemicals, pharmaceuticals, biologics, small organic molecules, antibodies, nucleic acids, peptides, and proteins. It is contemplated that these agents would be ingested by, or administered a subject which is ingesting the food or food product and would be ingested or administered before, during, or slightly after the ingestion of a food or food product in which the salty taste is to be modulated.

Another embodiment of the current invention is a composition comprising an agent that modulates the activation and/or activity of PKD2L1-expressing taste receptor cells and their concomitant pathway, in combination with NaCl. The concentration of NaCl can vary.

Another embodiment is a composition comprising an agent that modulates the activation and/or activity of PKD2L1-expressing taste receptor cells and their concomitant pathway, in combination with a sodium substitute, including but not limited to potassium chloride, magnesium chloride, and calcium chloride.

Another embodiment of the present invention is an agent or composition that modulates the activation and/or activity of PKD2L1-expressing taste receptor cells and their concomitant pathway added to a food or food products in which high-salt taste is desired, including but not limited to, crackers, potato chips, corn chips, tortilla chips, sauces, and canned soups and vegetables. These compositions will modulate the salty taste of the food or food product that contains either sodium (NaCl), or a sodium substitute (e.g., KCl).

A food or food product in which a high salt taste is desired and containing either sodium (NaCl) or a sodium substitute, and an agent or composition that modulates the activation and/or activity of PKD2L1-expressing taste receptor cells and their concomitant pathway are also part of the current invention. In a preferred embodiment the sodium substitute is potassium chloride.

It will be understood by those of skill in the art that one aspect of the invention allows the use of less sodium and/or a sodium substitute in a food or food product where a salty taste is desirable. This can be accomplished by modulating the function of the PKD2L1-expressing TRCs such that the subject perceives high salt where there is a lower amount of sodium and/or a sodium substitute. Thus, because less sodium or a substitute can be used in the food or food product, and the same desirable salty taste obtained, the food or food product is healthier but still satisfies the subject's desire for high salt taste.

It will also be understood by those of skill in the art that the invention allows the use of less sodium by using sodium substitutes and modulating the activity of PKD2L1-expressing TRCs in a food or food product where a salty taste is desirable. This can be accomplished by simultaneously modulating the function of the PKD2L1-expressing TRCs that normally respond to high salt such that the subject perceives high salt with the use of a substitute, but without the aversive responses associated with high concentrations of sodium and salt substitutes. Thus, because less sodium, i.e., a sodium substitute, can be used in the food or food product, without the substitute evoking a bad aftertaste, and the same desirable salty taste obtained, the food or food product is healthier but still satisfies the subject's desire for high salt taste.

Because high-salt activates both the bitter and PKD2L1-expressing taste receptor cells, the third method for modulating perceived salty taste is to alter the activation and/or activity of bitter-sensing taste receptor cells and their concomitant pathway, and the activation and/or activity of PKD2L1-expressing taste receptor cells and their concomitant pathway. This method can be accomplished by any agent or agents or composition or compositions that would modulate the activation, expression and/or action of a molecule or the receptor of the molecule in the pathways. Such agents include but are not limited to chemicals, phytochemicals, pharmaceuticals, biologics, small organic molecules, antibodies, nucleic acids, peptides, and proteins. Such compositions may contain one or more agents that alone or together, can modulate both the activation of the bitter-sensing TRCs and the PKD2L1-expressing TRCs. It is contemplated that these agents would be ingested by, or administered a subject which is ingesting the food or food product and would be ingested or administered before, during, or slightly after the ingestion of a food or food product in which the salty taste is to be modulated. Another embodiment of the current invention is a composition comprising an agent or agents that modulates both the activation and/or activity of bitter-sensing taste receptor cells and their concomitant pathway, and the activation and/or activity of PKD2L1-expressing taste receptor cells and their concomitant pathway, in combination with NaCl. The concentration of NaCl can vary.

Another embodiment is a composition comprising an agent or agents that modulates both the activation and/or activity of bitter-sensing taste receptor cells and their concomitant pathway, and the activation and/or activity of PKD2L1-expressing taste receptor cells and their concomitant pathway, in combination with a sodium substitute, including but not limited to potassium chloride, magnesium chloride, and calcium chloride,

Another embodiment of the present invention is an agent or agents or composition or compositions that modulates both the activation and/or activity of bitter-sensing taste receptor cells and their concomitant pathway, and the activation and/or activity of PKD2L1-expressing taste receptor cells and their concomitant pathway added to a food or food products in which high-salt taste is desired, including but not limited to, crackers, potato chips, corn chips, tortilla chips, sauces, and canned soups and vegetables. These agents or compositions will modulate the salty taste of the food or food product that contains either sodium (NaCl), or a sodium substitute (e.g., KCl).

A food or food product in which high salt taste is desired and containing either sodium (NaCl) or a sodium substitute, and an agent or agents or composition or compositions that modulates both the activation and/or activity of bitter-sensing taste receptor cells and their concomitant pathway, and the activation and/or activity of PKD2L1-expressing taste receptor cells and their concomitant pathway are also part of the current invention. In a preferred embodiment the sodium substitute is potassium chloride.

It will be understood by those of skill in the art that one aspect of the invention allows the use of less sodium and/or a sodium substitute in a food or food product where a salty taste is desirable. This can be accomplished by modulating the function of the bitter-sensing TRCs and the PKD2L1-expressing TRCs such that the subject perceives high salt where there is a lower amount of sodium and/or a sodium substitute. Thus, because less sodium or a substitute can be used in the food or food product, and the same desirable salty taste obtained, the food or food product is healthier but still satisfies the subject's desire for high salt taste.

It will also be understood by those of skill in the art that the invention allows the use of less sodium by using sodium substitutes and modulating the activity of bitter-sensing and PKD2L1-expressing TRCs in a food or food product where a salty taste is desirable. This can be accomplished by simultaneously modulating the function of the bitter-sensing and PKD2L1-expressing TRCs that normally respond to high salt such that the subject perceives high salt with the use of a substitute, but without the aversive responses associated with high concentrations of sodium and salt substitutes. Thus, because less sodium, i.e., a sodium substitute, can be used in the food or food product, without the substitute evoking a bad aftertaste, and the same desirable salty taste obtained, the food or food product is healthier but still satisfies the subject's desire for high salt taste.

Because carbonic anhydrases, in particular, CA4, play a role in the activation of the PKD2L1-expressing TRCs, another approach to modulating perceived salty taste is modulating the activation or action of, carbonic anhydrases, in particular CA4. Thus, a further method for modulating perceived salty taste is to alter the activation and/or activity of carbonic anhydrases, in particular CA4. This method can be accomplished by any agent that would modulate the activation and/or action of carbonic anhydrases, in particular CA4 molecule. Such agents include but are not limited to chemicals, phytochemicals, pharmaceuticals, biologics, small organic molecules, antibodies, nucleic acids, peptides, and proteins. Agents that modulate this activation include but are not limited to, dorzolamide (DZA), and benzolamide (BZA). It is contemplated that these agents would be ingested by, or administered a subject which is ingesting the food or food product and would be ingested or administered before, during, or slightly after the ingestion of a food or food product in which the salty taste is to be modulated.

Another embodiment of the current invention is a composition comprising an agent that modulates the activation and/or activity of carbonic anhydrases, in particular CA4 molecule, alone or in combination with NaCl. The concentration of NaCl can vary.

Another embodiment is a composition comprising an agent that modulates the activation and/or activity of carbonic anhydrases, in particular CA4 molecule, alone or in combination with a sodium substitute, including but not limited to potassium chloride, magnesium chloride, and calcium chloride.

Another embodiment of the present invention is an agent or composition that modulates the activation and/or activity of carbonic anhydrases, in particular CA4 molecule added to a food or food products in which high-salt taste is desired, including but not limited to, crackers, potato chips, corn chips, tortilla chips, sauces, and canned soups and vegetables. These compositions will modulate the salty taste of the food or food product that contains either sodium (NaCl), or a sodium substitute (e.g., KCl).

A food or food product containing either sodium (NaCl), or a sodium substitute, and an agent or composition that modulates the activation and/or activity of carbonic anhydrases, in particular CA4 molecule, is also part of the current invention. One such embodiment is a food or food product in which high salt is desired comprising either NaCl or a sodium substitute and DZA or BZA. In a preferred embodiment the sodium substitute is potassium chloride

It will be understood by those of skill in the art that one aspect of the invention allows the use of less sodium and/or a sodium substitute in a food or food product where salty taste is desirable. This can be accomplished by modulating the carbonic anhydrases, in particular the CA4 molecule, such that the subject perceives high salt where there is a lower amount of sodium and/or a sodium substitute. Thus, because less sodium and/or a substitute can be used in the food or food product, and the same desirable salty taste obtained, the food or food product is healthier but still satisfies the subject's desire for high salt taste.

It will also be understood by those of skill in the art that the invention allows the use of less sodium by using sodium substitutes and modulating the carbonic anhydrases, in particular the CA4 molecule, in a food or food product where a salty taste is desirable. This can be accomplished by simultaneously modulating the function the carbonic anhydrases, in particular the CA4 molecule, that normally respond to high salt such that the subject perceives high salt with the use of a substitute, but without the aversive responses associated with high concentrations of sodium and salt substitutes. Thus, because less sodium, i.e., a sodium substitute, can be used in the food or food product, without the substitute evoking a bad aftertaste, and the same desirable salty taste obtained, the food or food product is healthier but still satisfies the subject's desire for high salt taste.

A further approach to modulating perceived salty taste is accomplished by changing the pH of a product that is originally in an acidic environment, i.e., below 7, to a neutral environment, i.e., at or about 7, upon contact with the tongue, saliva, or oral cavity. This change in pH would prevent the activation of the CA4 on the PKD2L1-expressing TRCs by rendering the product pH neutral. Such a method would be accomplished by the addition of a composition to the product directly that could raise the pH to neutral, i.e., from below 7 to at or about 7, upon delivery of the product, i.e., the contact of the product with the tongue, saliva or oral cavity. This method could also be accomplished by encapsulating or packaging of the product such that the pH stays acidic until the product is contacted with the tongue, saliva or oral cavity, upon which the encapsulation or packaging allows the product to become a neutral pH.

Because the methods of use of the present invention are best accomplished via the product itself, food and food products which contain compositions that would modulate the activation of the bitter- and/or the PKD2L1-expressing taste receptor cells and/or their concomittant pathway, are also contemplated by the invention. Food and food products that contain compositions that raise the pH from acidic to neutral, or are composed or packaged in such a way that upon contact with the tongue, saliva or oral cavity, the pH is raised from acidic to neutral, are also contemplated by the invention.

### Screening Methods and Assays

Further embodiments of the present invention include screening methods and assays for identifying compounds or agents that would modulate perceived salty taste by altering or modulating the activity or activation of the bitter-sensing or PKD2L1-expressing TRCs and their pathways.

Such screening methods and assays include the use of methods that record the neural responses of an animal to various tastants, including but not limited to sodium at various concentrations, including low (less than 100 mM), medium (about 100 mM) and high (greater than 150 mM) concentrations; sodium substitutes including KCl; bitter; and sour. In particular, the neural responses of the animal to tastants such as sodium at various concentrations or sodium substitutes, and bitter or sour tastants is recorded before and after stimulation or contact with a test agent. If the neural responses to the tastants is changed or altered by the agent, it is an agent that can be used modulate perceived salty taste.

Potassium chloride can be used as a tastant in the methods and assays of the invention in an amount ranging from 0.03 to 1 M. Additionally, magnesium chloride and calcium chloride can also be used in these amounts as sodium substitute tastants.

Bitter tastants include but are not limited to, cycloheximide, denatonium, and quinine. These tastants can be used at concentration of about 0.1 to 10 mM.

Sour tastants include but are not limited to, citric acid. Citric acid can be used in concentrations of about 10 to 25 mM with 20 mM being preferred.

Methods that record neural responses are known in the art, and include but are not limited to, those described in Example 1, wherein the chorda tympani responses are recorded. Any animal can be used in the screening method or assay. Mammals are preferred, and mice are most preferred.

A further screening method or assay would include behavioral assays, which measure an animal's response, either attraction or aversion, to tastants, including but not limited to sodium at low (less than 100 mM), medium (about 100 mM), and high (greater than 150 mM) concentrations, sodium substitutes including KCl, bitter, and sour. Behavioral assays are known in the art and would include, but are not limited to, those described in Example 1. Any animal can be used in the screening method or assay. Mammals are preferred, and mice are most preferred.

In particular, the behavioral responses of the animal to tastants such as sodium at various concentrations or sodium substitutes, is recorded before and after stimulation or contact with a test agent. If the behavioral responses to the tastants is changed or altered by the agent, it is an agent that can be used modulate perceived salty taste.

Potassium chloride can be used as a tastant in the methods and assays of the invention in an amount ranging from 0.03 to 1 M. Additionally, magnesium chloride and calcium chloride can also be used in these amounts as sodium substitute tastants.

Bitter tastants include but are not limited to, cycloheximide, denatonium, and quinine. These tastants can be used at concentration of about 0.1 to 10 mM.

Sour tastants include but are not limited to, citric acid. Citric acid can be used in concentrations of about 10 to 25 mM with 20 mM being preferred.

A further embodiment is a screening method and/or assay that would screen for test compounds or agents that would modulate perceived salty taste by altering or modulating the activation of carbonic anhydrases, especially CA4.

Such screening methods and assays include the use of methods that record the neural responses of an animal to various tastants, including but not limited to sodium at various concentrations, including low (less than 100 mM), medium (about 100 mM) and high (greater than 150 mM) concentrations; and sodium substitutes including KCl, as well as an agent or composition that has a pH of less than 7.0. In particular, the neural responses of the animal to tastants such as sodium at various concentrations or sodium substitutes, and the agent or composition that has a pH of less than 7.0 are recorded before and after stimulation or contact with a test agent. If the neural responses to the tastants is changed or altered by the test agent, it is an agent that can be used modulate perceived salty taste.

Potassium chloride can be used as a tastant in the methods and assays of the invention in an amount ranging from 0.03 to 1 M. Additionally, magnesium chloride and calcium chloride can also be used in these amounts as sodium substitute tastants.

Methods that record neural responses are known in the art, and include but are not limited to, those described in Example 1, wherein the chorda tympani responses are recorded. Any animal can be used in the screening method or assay. Mammals are preferred, and mice are most preferred.

Such compounds or agents identified by any of these screening methods and assays as being useful to modulate the perceived taste of salt are also an embodiment of the invention.

### EXAMPLES

The present invention may be better understood by reference to the following non-limiting examples, which are presented in order to more fully illustrate the preferred embodiments of the invention. They should in no way be construed to limit the broad scope of the invention.

### Example 1

### Materials and Methods

Mice

All procedures followed the NIH Guidelines for the care and use of laboratory animals, and were approved by the Columbia University or National Institute of Dental and Craniofacial Research Animal Care and Use Committees.

T2R32-Sapphire mice are transgenics engineered to express the blue shifted GFP-derivative, Sapphire28, under the control of the T2R32 (also referred to as Tas2R139, PubMed gene #NM_{—}181275.1) promoter. These mice, generated by Ken Mueller (UCSD Thesis, 2004), contained 10 kbp upstream of the T2R32 start codon fused to the GFP reporter.

All other mouse strains have been described previously (Mueller et al. (2005); Zhang et al. (2003); Huang et al. (2006); Chandrashekar et al. (2009)).

Calcium Imaging

Calcium imaging from fungiform TRCs was performed as previously described (Chandrashekar et al. (2010); Oka et al. (2006)).

Fungiform TRCs were loaded in vivo with Calcium Green-1 dextran 3 kD (Invitrogen) by electroporating individual taste buds. Tongues were removed 24-36 hours after dye loading, and the epithelium was peeled enzymatically and placed in a custom recording chamber. The apical surface of the preparation was bathed in a constant flow of artificial saliva, and taste stimuli were delivered by focal application to individual taste buds. Tastants were applied for 1 second, with a minimum of 10 seconds of artificial saliva between stimuli. Changes in [Ca2+]i were monitored using a 5-Live confocal microscope (Zeiss) equipped with a ×40 C-Apochromat 1.20 W objective; images were captured at 4 Hz, and ΔF/F from individual TRCs analyzed and pseudo-colored as described previously (Chandrashekar et al. (2010)).

To identify sapphire positive cells in T2R32-Sapphire mice, 405-nm excitation laser to separate sapphire and Calcium Green-1 fluorescence was used. Mean cellular fluorescence intensity (F) was calculated for the individual TRCs and basal fluorescence (Fo) was assigned to each cell by averaging fluorescence intensity over 3 seconds just before tastant application. ΔF/F was calculated as [F−Fo]/Fo; taste cells were considered responders when ΔF/F exceeded 3 standard deviations above Fo within 5 seconds of tastant application.

Nerve (Chorda Tympani) Recordings

Lingual stimulation and recording procedures were performed as previously described (Chandrashekar et al. (2010); Nelson et al. (2002)). Data analysis used the integrated response during the 5 seconds of tastant stimulation.

Compounds used for nerve recordings were the following:

0.03-1 M NaCl (with and without 10 μM amiloride) or 0.03-1 M KCl (salty);

20 mM acesulfameK (sweet);

50 mM monopotassium glutamate (MPG) plus 0.5 mM inosine monophosphate (IMP) (umami);

0.1 mM cycloheximide (bitter); and

20 mM citric acid (sour).

Responses to 20 mM citric acid (FIGS. 2 and 3), 60 mM NaCl (FIG. 8, 12, and 13), or 250 mM KCl before AITC application (FIG. 13) were used to normalize responses for each experimental series. For FIG. 7, data were normalized to 20 mM citric acid and then scaled to WT responses before AITC application. Data were analyzed for statistical significance using an unpaired, one-tailed Student's t-test and 95% confidence limits.

To compute the amiloride-sensitive salt component, the stimulation regime involved sequential applications of NaCl solutions first without, and then with, amiloride (5 seconds pre- or pre- and post-incubation and co-application with NaCl solution) in the same experimental series. The amiloride-insensitive component was defined as the response in the presence of amiloride. The fraction of the response inhibited by amiloride was defined as the amiloride-sensitive component (amiloride-sensitive component=response without amiloride−response with amiloride).

For pharmacological inhibition studies using allyl isothiocyanate (AITC), responses to a series of taste stimuli were measured. Then 3 mM AITC (Aldrich, 377430-5G) was applied to the tongue at a rate of 6 ml/minute for 5 minutes. The tongue was washed with artificial saliva for 1 minute and nerve responses to the same series of taste stimuli measured. Responses before and after AITC were compared for each animal. To minimize effects of recovery, responses after AITC were recorded within 15 minutes of AITC treatment.

Behavioral Assays

Behavioral assays used a custom-made gustometer to measure immediate lick responses as described previously (Chandrashekar et al. (2010); Mueller et al. (2005); Zhang et al. (2003)).

For salt-attraction assays, mice were injected with furosemide (50 mg/kg) and were placed in their home cage for 3 hours, without food or water before testing.

For salt aversion assays, mice were water deprived for 24 hours before testing.

Three or four (attraction assay) or two (aversion assay) different concentrations of tastant and water were presented to animals in each experimental session. Differences between knockout and control mice were analyzed for statistical significance using a two-way ANOVA with a Bonferroni post hoc test.

### Example 2

### Characterization of T2R32-Sapphire Mice

Expression of the sapphire gene and other taste receptors in taste tissue of the T2R32-Sapphire mice was characterized.

Materials and Methods

Mice as described in Example 1 were used.

Results

Double label in situ hybridization showed the expression of the sapphire transgene in taste buds of T2R32-sapphire transgenic mice as well as the expression of taste receptors. As shown in FIG. 1A, Sapphire (left panel, red-label) and bitter taste receptors (a mix of 20 T2Rs, middle panel, green label) are extensively co-expressed (right panel, merged image). Quantitation of labeling through the circumvallate papilla of two T2R32-sapphire mice revealed that at least 75% of positive cells were strongly detected by both probes.

In contrast, FIGS. 1B and 1C show Sapphire (red) was never co-expressed with B. T1R3 (green), a component of sweet and umami receptors (Zhao et al. (2003)) or C. PKD1L3 (green), a marker of sour responsive cells (Huang et al. (2006)).

### Example 3

### Identification of a Pharmacological Blocker of the High-Salt Sensing Pathway

A pharmacological blocker of the high-salt sensing pathway was identified to be used as a tool to dissect the cellular basis of high-salt taste.

Materials and Methods

Mice as described in Example 1 were used.

The chorda tympani taste responses were recorded as described in Example 1.

Taste responses were recorded in the presence and absence of various compounds known to affect ion channel function as shown in Table 1.

| NAME OF CHEMICAL | CONCENTRATION | |
|---|---|---|
| Mefloquine | 10 | mM |
| Probenecid | 100 | mM |
| Chloroquine | 10 | mM |
| 2-APB | 3 | mM |
| Alpha-glycyrrhetinic acid | 2.5 | mM |
| Verapamil | 1 | mM |
| 4-AP | 100 | mM |
| Citric acid | 20 | mM |
| DIDS | 30 | mM |
| Flupiritine | 100% | |
| Carbenoxolone | 30 | mM |
| AITC | 1-10 | mM |
| Capsaicin | 100 | uM |
| Amiloride | 30 | uM |
| Garlic extract | 1% | |
| H3030031 (TRPA1 antagonist) | 200 | uM |
| AP-18 (TRPA1 antagonist) | 1 | mM |
| LaCl₃ | 3 | mM |

Results

It was found that allyl isothiocyanate (AITC) significantly suppressed high-sodium responses (FIG. 2 upper panel) without affecting responses to low concentrations of NaCl. Amiloride was used to selectively eliminate the contribution of the ENaC-dependent, low-salt pathways. AITC completely inhibited bitter responses (0.1 mM cycloheximide) and significantly suppressed high-salt (250 or 500 mM NaCl amiloride and KCl) responses (highlighted in red) but did not affect responses to low salt (60 mM NaCl) or other taste qualities (FIG. 2 and FIG. 3).

Identical suppression was observed for KCl, which selectively activates the high-salt pathway (FIG. 2 and FIG. 3).

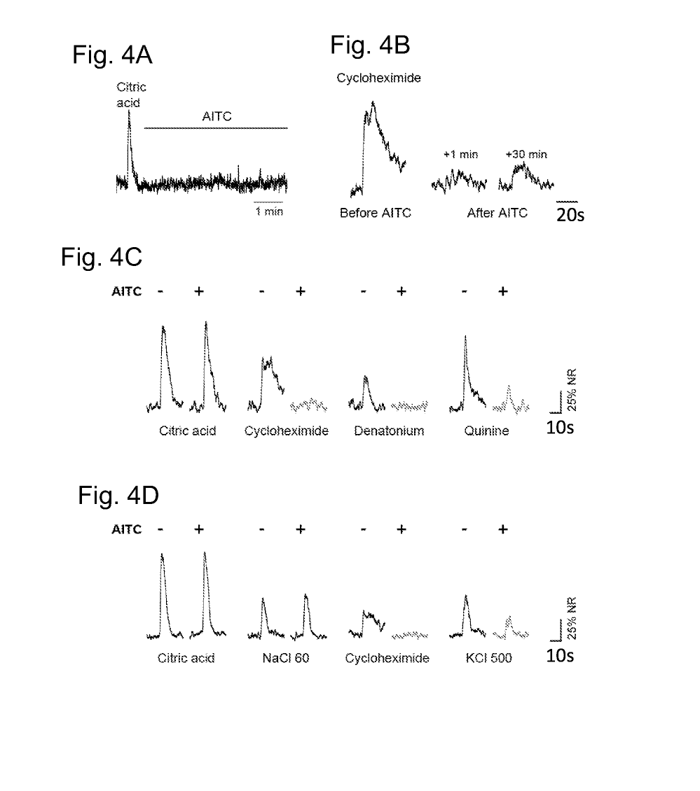

AITC also inhibited responses to bitter stimuli without significantly impacting any other taste modality. 5 minutes exposure of the tongue to 3 mM AITC evokes minimal taste response but AITC strongly suppresses responses to high concentrations of the bitter tastant cycloheximide (1 mM) at both 1 and 30 minutes after AITC treatment (FIGS. 4A and 4B). AITC treatment (indicated by + above the trace) not only suppressed responses to 0.1 mM cycloheximide but also to other bitter tastants, 10 mM denatonium, and 10 mM quinine (red traces), while sour taste (20 mM citric acid) remained unimpaired (FIG. 4C).

Additionally, TRPA1-KO mice exhibited robust AITC-mediated suppression of bitter and high-salt taste responses (red traces) (FIG. 4D). Thus, TRPA1 is noₜ required for inhibition of taste responses by AITC.

These results suggested that bitter taste receptor cells might be the target of AITC, and a constituent of the high-salt sensing pathway.

### Example 4

### Bitter-Sensing Taste Receptor Cells are Activated by High-Salt Stimuli

Materials and Methods

The T2R32-Sapphire mice as described in Examples 1 and 2 were used.

Calcium imaging as described in Example 1 was performed.

Results

It was found that in the T2R32-Sapphire mice, high concentrations of salt activated the GFP-positive cells, which in turn responded to bitter. T2R32-Sapphire positive taste cells responded to bitter stimuli (mixture of 1 mM cycloheximide, 1 mM quinine, and 10 mM denatonium) and high-salt (500 mM KCl) but not to sour stimuli (100 mM citric acid) (FIGS. 5 and 6). In total, 15 and 12 Sapphire-positive cells were activated by bitter and KCl respectively; among these, 11 cells were activated by both compounds, but not by sour stimuli.

### Example 5

### Aversion to High Salt Stimuli is Mediated in Part by Activation of the Bitter-Sensing Pathway

Based upon the observations that high salt activates bitter-sensing cells, and that high salt and bitter stimuli are both blocked by AITC, it was hypothesized that bitter and high salt may share a common pathway.

Materials and Methods

TRPM5 and PLCβ2 knockout (KO) mice (as described in Example 1 and Zhang et al. (2003)) were used.

The chorda tympani taste responses were performed and recorded as described in Example 1.

Results

The nerve responses to high salt of the TRPM5 and PLCβ2 knockout (KO), which lack key components for bitter taste signaling, was significantly reduced, and moreover, they were no longer sensitive to AITC (FIG. 7).

AITC and TRPM5/PLCβ2 knockouts eliminated only about 50% of the high-salt neural responses (FIG. 7). These animals still retained a strong behavioral aversion to high salt.

To rigorously demonstrate that the TRPM5- and PLCβ2-dependent high-salt responses are mediated by bitter receptor cells, a selective-rescue experiment was performed whereby PLC function was restored only to bitter taste receptor cells of PLCβ2 knockout mice. The expression of a wild-type PLC transgene in bitter receptor cells fully rescued the electrophysiological responses to both bitter and high-salt (KCl) to levels indistinguishable from those in wild type mice (FIG. 7A bottom panel and 7B).

AITC treatment almost completely suppressed responses to 0.1 mM cycloheximide and reduced by half the responses to 500 mM KCl and 500 mM NaCl in the presence of 10 μM amiloride in control and T2R-PLC rescue animals.

These results demonstrate that bitter sensing cells mediate the PLCP2-dependent high-salt responses, and support the proposal that the aversion to high-salt is mediated, at least in part, by activation of the bitter sensing pathway.

### Example 6

### Aversion to High Salt Stimuli is Mediated in Part by Activation of the Sour-Sensing Pathway

Materials and Methods

Mice as previously described were engineered by targeting the tetanus toxin light chain (TeNT) to their sour taste receptor cells (PKDL1-expressing cells), effectively silencing these cells (Huang et al. (2006); Chandrashekar et al (2009); Yamamoto et al. (2003)). These mice were designated PKD2L1-TeNT.

Double-mutant mice expressing PKD2L1-TeNT and harboring a TRPM5 mutation were also used. These mice had both sour taste receptor cell and bitter taste receptor cell pathways genetically blocked. These mice were designated TRPM5KO/PKD2L1-TeNT.

The chorda tympani taste responses were performed and recorded as described in Example 1.

Results

As shown previously (Chandrashekar et al. (2009), silencing PKD2L1-expressing cells eliminated acid-evoked taste responses (FIG. 8A).

Additionally, as shown in FIG. 8, these animals also displayed a major reduction in their high-salt electrophysiological responses, and further treatment with AITC effectively abolished their remaining high-salt (KCl) responses (FIG. 9).

Since it was hypothesized that that high-salt taste responses are most likely mediated by the combined action of bitter and sour-sensing cells, genetically blocking both pathways should abolish high-salt responses. Indeed, TRPM5KO/PKD2L1-TeNT double mutant mice exhibited a near complete loss of electrophysiological taste responses to a variety of high-salts (FIG. 8), including concentrations of NaCl as high as 1000 mM.

### Example 7

### Behavioral Aversion to High Salt

Since both the bitter- and sour-tasting cellular pathways are mediators of behavioral aversion to high-salt, then it was hypothesized that silencing both the pathways would abolish rejection of high-salt.

Materials and Methods

TRPM5-KO mice as described in Example 5, and PKD2L1-TeNT and TRPM5KO/PKD2L1-TeNT double mutant mice as described in Example 6 were used.

The behavior assays were performed as described in Example 1.

The chorda tympani taste responses were performed and recorded as described in Example 1.

Results

If these two cellular pathways are the mediators of behavioral aversion to high-salt, then simultaneously silencing both the T2R and PKD2L1-expressing cells should abolish rejection of concentrated salt solutions.

As shown in FIGS. 10A and 11, wild type mice exhibited robust dose dependent behavioral aversion to increasing concentrations of KCI, and single mutant mice, TRPM5−/− or PKD2L1-TeNT, behaved as wild-type mice and still retained strong aversion to high salt, demonstrating that activation of either pathway on its own is sufficient to trigger behavioral rejection to salt.

In contrast, TRPM5-KO/PKD2L1-TeNT double mutant animals did not avoid high-salt stimuli (FIG. 10A), even at concentrations where controls were strongly repelled. Remarkably, these double mutants were not simply indifferent to high-salt, but exhibited unimpeded attraction, even to exceedingly high concentrations of salt (e.g. levels equivalent to ocean water; approximately 500 mM NaCl; FIG. 10B).

After sodium depletion, wild type mice exhibited powerful attractive responses to NaCl but the attraction was considerably reduced at higher concentration (500 mM) (FIGS. 10B and 12). In contrast, double mutant animals showed a continuous increase in attraction even at concentrations as high as 500 mM NaCl.

### Example 8

### Inhibition of Tongue Carbonic Anhydrases Impairs High salt Sensing by Sour Taste Receptor Cells

Materials and Methods

Mice as described in Example 1 were used.

The chorda tympani taste responses were performed and recorded as described in Example 1.

AITC pretreatment was carried out to eliminate the bitter cell component of salt taste, as described in Example 1.

For pharmacological studies using bicarbonate, taste responses were measured in the presence or absence of 30 mM KHCO₃ (pH7.4) (5 second pre-incubation and co-application with stimuli). In dorzolamide (DZA) experiments, responses were monitored before and after incubation of the tongue with 0.5% DZA (w/v) for 5 minutes.

Results

Using knockout mice for CA4 it was shown that the inhibition of CA4 dramatically reduced high salt taste responses mediated by the sour sensing cells. The chorda tympani responses from control (WT), heterozygous CA4 +/−, and homozygous CA4 −/− mice showed that the homozygous CA4 −/− mice have a greatly reduced responses to high-salt (250 mM KCl) (FIG. 13A).

Driving CA4 in the direction that raises pH by addition of excess CA4 substrate (HCO₃−) also suppressed PKD2L1-cell mediated high-salt responses. TRPM5-KO mice were used to focus on responses from sour sensitive cells. The chorda tympani responses to 500 mM KCl or 100 mM CaCl₂ before and after addition of 30 mM KHCO₃ demonstrated strong inhibition of high salt taste by bicarbonate (FIG. 13B).

Pharmacological inhibition of tongue carbonic anhydrases with the potent CA inhibitor (dorzolamide, DZA, 0.5% w/v) also reduced high-salt induced chorda tympani responses from sour taste cells. The responses of TRPM5-KO mice to various salts, before and after treatment of the tongue with DZA, showed that high-salt (as well as CO₂) responses were affected by DZA treatment. As expected, sour (20 mM citric acid) and low salt (NaCl, 60 mM) taste responses were insensitive to DZA. The taste responses from PKD2L1-TeNT mice were completely insensitive to DZA treatment, demonstrating that inhibition of carbonic anhydrase activity does not affect high-salt responses in T2R-expressing bitter cells (FIG. 13C).

### Example 9

### Lowering the Salivary pH Enhances the High Salt Responses of Sour Taste Receptor Cells

If CA4 functions as a “translator” of external salt concentration into local pH changes, it was hypothesized that lowering salivary pH should enhance the high-salt responses of sour (but not bitter) cells.

Materials and Methods

TRPM5-KO mice as described in Example 5, and PKD2L1-TeNT mice as described in Example 6 were used.

The chorda tympani taste responses were performed and recorded as described in Example 1.

To study effects of pH on nerve responses the pH of artificial saliva (7.4) was adjusted to 5.5 with hydrochloric acid.

Results

The chorda tympani responses to 500 mM KCl and 100 mM CaCl₂ demonstrated that reducing pH from 7.4 (normal artificial saliva) to 5.5 significantly enhanced high-salt responses in TRPM5-KO mice (FIG. 14A).

As expected, high-salt responses were not affected by salivary pH in PKD2L1-TeNT mice, demonstrating that CA4 and PKD2L1-cell dependent high-salt responses but not those of bitter-cells are pH sensitive (FIG. 14B)

### REFERENCES

Baird et al. (1997) “Catalysis and inhibition of human carbonic anhydrase IV” Biochemistry 36:2669-78
Beauchamp et al. (1990) “Experimental sodium depletion and salt taste in normal human volunteers” Am. J. Clin. Nutr. 51:881-9
Breslin and Beauchamp (1995) “Suppression of bitterness by sodium: variation among bitter taste stimuli” Chem. Senses 20:609-23
Chandrashekar et al. (2006) “The receptors and cells for mammalian taste” Nature 444:288-94
Chandrashekar et al. (2009) “The taste of carbonation” Science 326:443-5
Chandrashekar et al. (2010) “The cells and peripheral representation of sodium taste in mice” Nature 464:297-301
Contreras (1989) “Gustatory mechanisms of a specific appetite” In: Neural Mechanisms in Taste, edited by Cagan R. H. Boca Raton, Fla.: CRC, 119-145
Dando and Roper (2012) “Acetylcholine is released from taste cells, enhancing taste signaling” J. Physiol. 590:3009-3107

Doolin and Gilbertson (1996) “Distribution and characterization of functional am iloridesensitive sodium channels in rat tongue” J Gen. Physiol. 107:545-54
Duncan (1962) “Salt preferences of birds and mammals” Physiol. Zool. 35:120-132
Eylam and Spector (2005) “Taste discrimination between NaCl and KCl is disrupted by amiloride in inbred mice with amiloride-insensitive chorda tympani nerves” Am. J. Physiol. Regul. Integr. Comp. Physiol. 288:R1361-8
Gao et al. (2000) “Site-directed mutagenesis studies of human A(2A) adenosine receptors: involvement of glu(13) and his(278) in ligand binding and sodium modulation” Biochem. Pharmacol. 60:661-8
Hallock et al. (2009) “Residual chemosensory capabilities in double P2X2/P2X3 purinergic receptor null mice: intraoral or postingestive detection?” Chem. Senses 34:799-808
Halpern (1998) “Amiloride and vertebrate gustatory responses to NaCl” Neurosci. Biobehay. Rev. 23:5-47
He and MacGregor (2009) “A comprehensive review on salt and health and current experience of worldwide salt reduction programmes” J. Hum. Hypertens. 23:363-84
Heck et al. (1984) “Salt taste transduction occurs through an amiloride-sensitive sodium transport pathway” Science 223:403-5
Hellekant et al (1997) “Taste in chimpanzees II: single chorda tympani fibers” Physiol. Behav. 61:829-41
Herness and Zhao (2009) “The neuropeptides CCK and NPY and the changing view of cell-to-cell communication in the taste bud.” Physiol. Behav. 97:581-591
Hettinger and Frank (1990) “Specificity of amiloride inhibition of hamster taste responses” Brain Res. 513:24-34
Huang et al. (2006) “The cells and logic for mammalian sour taste detection” Nature 442:934-8
Huang et al. (2012) “Glutamate may be an efferent transmitter that elicits inhibition in mouse taste buds” PLoS One 7(1):e30662
Institute of Medicine (2004) “Dietary reference intakes for water, potassium, sodium chloride, and sulfate” 1st ed. Washington, DC: The National Academies Press
Lindemann (2001) “Receptors and transduction in taste” Nature 413:219-25
Liu et al. (2012) “Structural basis for allosteric regulation of GPCRs by sodium ions” Science 337:232-6
Mueller et al. (2005) “The receptors and coding logic for bitter taste” Nature 434:225-9
Nelson et al. (2002) “An amino-acid taste receptor” Nature 416:199-202
Ohkuri et al. (2012) “Residual Chemoresponsiveness to Acids in the Superior Laryngeal Nerve in ‘Taste-Blind’ (P2X2/P2X3 Double-KO) Mice” Chem. Senses 37:523-32
Oka et al. (2006) “Odorant receptor map in the mouse olfactory bulb: in vivo sensitivity and specificity of receptor-defined glomeruli” Neuron 52:857-69
Roper (2007) “Signal transduction and information processing in mammalian taste buds.” Pflugers Arch. 454:759-776
Spector et al. (1996) “Amiloride disrupts NaCl versus KCl discrimination performance: implications for salt taste coding in rats” J. Neurosci, 16:8115-22
Ugawa et al (2005) “Coexpression of vanilloid receptor subtype-1 and acid-sensing ion channel genes in the human trigeminal ganglion neurons” Chem. Senses 30 Suppl 1:i195
Vasanser et al. (2002) “Residual lifetime risk for developing hypertension in middle-aged women and men: The Framingham Heart Study” JAMA 287:1003-10
Yamamoto et al. (2003) “Reversible suppression of glutamatergic neurotransmission of cerebellar granule cells in vivo by genetically manipulated expression of tetanus neurotoxin light chain” J. Neurosci. 23: 6759-67
Yarmolinsky et al. (2009) “Common sense about taste: from mammals to insects” Cell 139:234-44
Zhang et al. (2003) “Coding of sweet, bitter, and umami tastes: different receptor cells sharing similar signaling pathways” Cell 112:293-301
Zhao et al. (2003) “The receptors for mammalian sweet and umami taste” Cell 115:255-66
Zhu and Sly (1990) “Carbonic anhydrase IV from human lung: Purification, characterization, and comparison with membrane carbonic anhydrase from human kidney” J. Biol. Chem. 265:8795-801

1. A method for modulating the perceived saltiness of a salty taste stimulant in a food or food product, comprising administering to a subject who is ingesting the food or food product, an agent that alters the activation or activity of bitter-sensing taste receptor cells (TRCs) and/or their concomitant pathway.

2. The method of claim 1, wherein the agent is administered before, during or slightly after the subject ingests the food or food product.

3. The method of claim 1, wherein the food or food product contains sodium chloride.

4. The method of claim 1, wherein the food or food product contains a sodium substitute.

5. The method of claim 4, wherein the sodium substitute is chosen from the group consisting of potassium chloride, magnesium chloride, and calcium chloride.

6. The method of claim 1, wherein the food or food product is chosen from the group consisting of crackers, potato chips, corn chips, tortilla chips, sauces, canned soups, and canned vegetables.

7. The method of claim 1, wherein the agent is chosen from the group consisting of chemicals, phytochemicals, pharmaceuticals, biologics, small organic molecules, antibodies, nucleic acids, peptides, and proteins.

8. The method of claim 1, wherein the agent is allyl isothiocyanate.

9. A composition for modulating the perceived saltiness of a salty taste stimulant in a food or food product, comprising an agent that alters the activation or activity of bitter-sensing taste receptor cells (TRCs) and/or their concomitant pathway.

10. The composition of claim 9, further comprising sodium chloride.

11. The composition of claim 9, further comprising a sodium substitute.

12. The composition of claim 11, wherein the sodium substitute is chosen from the group consisting of potassium chloride, magnesium chloride, and calcium chloride.

13. The composition of claim 9, wherein the agent is allyl isothiocyanate.

14. A food or food product comprising sodium chloride or a sodium substitute and an agent that alters the activation or activity of bitter-sensing taste receptor cells (TRCs) and/or their concomitant pathway.

15. The food or food product of claim 14, wherein the sodium substitute is chosen from the group consisting of potassium chloride, magnesium chloride, and calcium chloride.

16. The food or food product of claim 14, wherein the food or food product is chosen from the group consisting of crackers, potato chips, corn chips, tortilla chips, sauces, and canned soups and vegetables.

17. The food or food product of claim 14, wherein the agent is chosen from the group consisting of chemicals, phytochemicals, pharmaceuticals, biologics, small organic molecules, antibodies, nucleic acids, peptides, and proteins.

18. The food or food product of claim 14, wherein the agent is allyl isothiocyanate.

19. A method for modulating the perceived saltiness of a salty taste stimulant in a food or food product, comprising administering to a subject who is ingesting the food or food product, an agent that alters the activation or activity of PKD2L1-expressing taste receptor cells (TRCs) and/or their concomitant pathway.

20. The method of claim 19, wherein the agent is administered before, during or slightly after the subject ingests the food or food product.

21. The method of claim 19, wherein the food or food product contains sodium chloride.

22. The method of claim 19, wherein the food or food product contains a sodium substitute.

23. The method of claim 22, wherein the sodium substitute is chosen from the group consisting of potassium chloride, magnesium chloride, and calcium chloride.

24. The method of claim 19, wherein the food or food product is chosen from the group consisting of crackers, potato chips, corn chips, tortilla chips, sauces, canned soups and canned vegetables.

25. The method of claim 19, wherein the agent is chosen from the group consisting of chemicals, phytochemicals, pharmaceuticals, biologics, small organic molecules, antibodies, nucleic acids, peptides, and proteins.

26. A composition for modulating the perceived saltiness of a salty taste stimulant in a food or food product, comprising an agent that alters the activation or activity of PKD2L1-expressing taste receptor cells (TRCs) and/or their concomitant pathway.

27. The composition of claim 26, further comprising sodium chloride.

28. The composition of claim 26, further comprising a sodium substitute.

29. The composition of claim 28, wherein the sodium substitute is chosen from the group consisting of potassium chloride, magnesium chloride, and calcium chloride.

30. A food or food product comprising sodium chloride or a sodium substitute and an agent that alters the activation or activity of PKD2L1-expressing taste receptor cells (TRCs) and/or their concomitant pathway.

31. The food or food product of claim 30, wherein the sodium substitute is chosen from the group consisting of potassium chloride, magnesium chloride, and calcium chloride.

32. The food or food product of claim 30, wherein the food or food product is chosen from the group consisting of crackers, potato chips, corn chips, tortilla chips, sauces, canned soups, and canned vegetables.

33. The food or food product of claim 30, wherein the agent is chosen from the group consisting of chemicals, phytochemicals, pharmaceuticals, biologics, small organic molecules, antibodies, nucleic acids, peptides, and proteins.

34. A method for modulating the perceived saltiness of a salty taste stimulant in a food or food product, comprising administering to a subject who is ingesting the food or food product, an agent that alters the activation or activity of carbonic anhydrase 4 (CA4).

35. The method of claim 34, wherein the agent is administered before, during or slightly after the subject ingests the food or food product.

36. The method of claim 34, wherein the food or food product contains sodium chloride.

37. The method of claim 34, wherein the food or food product contains a sodium substitute.

38. The method of claim 37, wherein the sodium substitute is chosen from the group consisting of potassium chloride, magnesium chloride, and calcium chloride.

39. The method of claim 34, wherein the food or food product is chosen from the group consisting of crackers, potato chips, corn chips, tortilla chips, sauces, and canned soups and vegetables.

40. The method of claim 34, wherein the agent is chosen from the group consisting of chemicals, phytochemicals, pharmaceuticals, biologics, small organic molecules, antibodies, nucleic acids, peptides, and proteins.

41. The method of claim 34, wherein the agent is chosen from the group consisting of dorzolamide (DZA), and benzolamide (BZA).

42. A composition for modulating the perceived saltiness of a salty taste stimulant in a food or food product, comprising an agent that alters the activation or activity of carbonic anhydrase 4 (CA4).

43. The composition of claim 42, further comprising sodium chloride.

44. The composition of claim 42, further comprising a sodium substitute.

45. The composition of claim 44, wherein the sodium substitute is chosen from the group consisting of potassium chloride, magnesium chloride, and calcium chloride.

46. The composition of claim 42, wherein the agent is chosen from the group consisting of dorzolamide (DZA), and benzolamide (BZA).

47. A food or food product comprising sodium chloride or a sodium substitute and an agent that alters the activation or activity of carbonic anhydrase 4 (CA4).

48. The food or food product of claim 47, wherein the sodium substitute is chosen from the group consisting of potassium chloride, magnesium chloride, and calcium chloride.

49. The food or food product of claim 47, wherein the food or food product is chosen from the group consisting of crackers, potato chips, corn chips, tortilla chips, sauces, canned soups, and canned vegetables.

50. The food or food product of claim 47, wherein the agent is chosen from the group consisting of chemicals, phytochemicals, pharmaceuticals, biologics, small organic molecules, antibodies, nucleic acids, peptides, and proteins.

51. The food or food product of claim 47, wherein the agent is chosen from the group consisting of dorzolamide (DZA), and benzolamide (BZA).

52. A method for modulating the perceived saltiness of a salty taste stimulant in a food or food product, comprising administering to a subject who is ingesting the food or food product, an agent or agents that alters the activation or activity of PKD2L1-expressing taste receptor cells (TRCs) and/or their concomitant pathway, and the activation or activity of bitter-sensing taste receptor cells (TRCs) and/or their concomitant pathway.

53. The method of claim 52, wherein the agent or agents is administered before, during or slightly after the subject ingests the food or food product.

54. The method of claim 52, wherein the food or food product contains sodium chloride.

55. The method of claim 52, wherein the food or food product contains a sodium substitute.

56. The method of claim 55, wherein the sodium substitute is chosen from the group consisting of potassium chloride, magnesium chloride, and calcium chloride.

57. The method of claim 52, wherein the food or food product is chosen from the group consisting of crackers, potato chips, corn chips, tortilla chips, sauces, canned soups, and canned vegetables.

58. The method of claim 52, wherein the agent or agents is chosen from the group consisting of chemicals, phytochemicals, pharmaceuticals, biologics, small organic molecules, antibodies, nucleic acids, peptides, and proteins.

59. A composition for modulating the perceived saltiness of a salty taste stimulant in a food or food product, comprising an agent or agents that alters the activation or activity of PKD2L1-expressing taste receptor cells (TRCs) and/or their concomitant pathway, and bitter-sensing taste receptor cells (TRCs) and/or their concomitant pathway.

60. The composition of claim 59, further comprising sodium chloride.

61. The composition of claim 59, further comprising a sodium substitute.

62. The composition of claim 59, wherein the sodium substitute is chosen from the group consisting of potassium chloride, magnesium chloride, and calcium chloride.

63. A food or food product comprising sodium chloride or a sodium substitute and an agent that alters the activation or activity of PKD2L1-expressing taste receptor cells (TRCs) and/or their concomitant pathway and bitter-sensing taste receptor cells (TRCs) and/or their concomitant pathway.

64. The food or food product of claim 63, wherein the sodium substitute is chosen from the group consisting of potassium chloride, magnesium chloride, and calcium chloride.

65. The food or food product of claim 63, wherein the food or food product is chosen from the group consisting of crackers, potato chips, corn chips, tortilla chips, sauces, and canned soups and vegetables.

66. The food or food product of claim 63, wherein the agent is chosen from the group consisting of chemicals, phytochemicals, pharmaceuticals, biologics, small organic molecules, antibodies, nucleic acids, peptides, and proteins.

67. A method for identifying an agent for modulating the perception of salty taste in a food or food product by altering the activation or activity of bitter-sensing taste receptor cells (TRCs) and/or their concomitant pathway, comprising:
a. stimulating the tongue of an animal with a high concentration of sodium chloride or sodium substitute, wherein neural responses to taste of the animal can be recorded;
b. recording the neural response of the animal to the high concentration of sodium chloride or sodium substitute;
c. stimulating the tongue of an animal with a bitter taste stimulant, wherein neural responses to taste of the animal can be recorded;
d. recording the neural response of the animal to the bitter taste stimulant;
e. administering the agent to the animal;
f. repeating steps a.-d.; and
g. comparing the second neural responses of the animal to the first neural responses of the animal;
wherein if the second neural responses of the animal to the sodium chloride or the sodium substitute, and the bitter taste stimulant, are less than the first neural responses of the animal to the sodium chloride or the sodium substitute, and the bitter taste stimulant, the agent is altering or modulating the activation or activity of bitter-sensing taste receptor cells (TRCs) and/or their concomitant pathway by the high concentration of sodium or the sodium substitute, and can be used to modulate the perception of salty taste in a food or food product.

68. A method for identifying an agent for modulating the perception of salty taste in a food or food product by altering the activation or activity of PKD2L1-expressing taste receptor cells (TRCs) and/or their concomitant pathway, comprising:
a. stimulating the tongue of an animal with a high concentration of sodium chloride or sodium substitute, wherein neural responses to taste of the animal can be recorded;
b. recording the neural response of the animal to the high concentration of sodium chloride or sodium substitute;
c. stimulating the tongue of the animal with an acid taste stimulant, wherein neural responses to taste of the animal can be recorded;
d. recording the neural response of the animal to the acid taste stimulant;
e. administering the agent to the animal;
f. repeating steps a.-d.; and
g. comparing the second neural responses of the animal to the first neural responses;
wherein if the second neural responses of the animal to the sodium chloride or the sodium substitute, and the acid taste stimulant, are less than the first neural responses of the animal to the sodium chloride or the sodium substitute and the acid taste stimulant, the agent is altering or modulating the activation or activity of PKD2L1-expressing taste receptor cells (TRCs) and/or their concomitant pathway by the high concentration of sodium or the sodium substitute, and can be used to modulate the perception of salty taste in a food or food product.

69. A method for identifying agents that modulate the attraction or aversion to high concentration of sodium or sodium substitutes, comprising:
a. treating a cohort of animals to conditions favoring salt-aversion or salt attraction;
b. administering the agent to some of the cohort of animals;
c. recording the behavior of the entire cohort of animals in response to sodium or sodium substitutes; and
d. comparing the aversion or attraction to the sodium or sodium substitutes of the animals administered the agent, to those animals who were not administered the agent;
wherein if the animals which received the agent have a different behavioral response to the sodium or sodium substitute, the agent is modulating the attraction or aversion to sodium or sodium substitutes.

70. A method for identifying agents for modulating the perception of salty taste stimulant in a food or food product that alter the activation and/or action of the enzyme CA4, or those which modulate the activity of PKD2L1-expressing TRCs comprising:
a. stimulating the tongue of an animal with a high concentration of sodium chloride or sodium substitute, wherein neural responses to taste of the animal can be recorded;
b. recording the neural response of the animal to the high concentration of sodium chloride or sodium substitute;
c. stimulating the tongue of the animal with an agent or composition with a pH of less than 7.0, wherein neural responses to taste of the animal can be recorded;
d. recording the neural response of the animal to the agent or composition with a pH of less than 7.0;
e. administering the test agent to the animal;
f. repeating steps a.-d.; and
g. comparing the second neural responses of the animal to the first neural responses;
wherein if the second neural responses of the animal to the sodium chloride or the sodium substitute, and the agent or composition with a pH of less than 7.0, are less than the first neural responses of the animal to the sodium chloride or the sodium substitute and the agent or composition with a pH of less than 7.0, the test agent is altering the activation and/or action of the enzyme CA4, by the high concentration of sodium or the sodium substitute, and can be used to modulate the perception of salty taste in a food or food product.
